(19) United States
(12) Patent Application Publication
(10) Pub. No.: US 2025/0387371 A1
(43) Pub. Date: Dec. 25, 2025

(54) COMPOSITIONS AND FORMULATION METHODS FOR SUSTAINED LOCAL RELEASE OF ANTIFIBROTICS

(71) Applicant: THE HOPKINS UNIVERISTY, BALTIMORE, MD (US)

(72) Inventors: Laura ENSIGN-HODGES, Towson, MD (US); Florin M. SELARU, Clarksville, MD (US); Min Kyung JOO, Baltimore, MD (US); Rachel SHAPIRO, Baltimore, MD (US); Ling LI, Baltimore, MD (US)

(21) Appl. No.: 18/880,412

(22) PCT Filed: Jul. 11, 2023
(86) PCT No.: PCT/US2023/027375

§ 371 Date: Dec. 31, 2024

Related U.S. Application Data
(60) Provisional application No. 63/388,120, filed on Jul. 11, 2022.

(51) Int. Cl. A61K 31/4174; A61K 9/00; A61K 9/14; A61K 47/02; A61K 47/10; A61K 47/32; A61P 1/00

(52) U.S. Cl. CPC A61K 31/4174; A61K 9/0019; A61K 9/146; A61K 47/02; A61K 47/10; A61K 47/32; A61P 1/00

(57) ABSTRACT

Methods for creating injectable sustained release nanocrystals to deliver antifibrotic drugs in a sustained fashion locally in tissue are described.

### FEDERALLY SPONSORED RESEARCH OR DEVELOPMENT

This invention was made with government support under grants CA190040, DK107806 and EB017742 awarded by the National Institutes of Health. The government has certain rights in the invention.

### BACKGROUND

Abnormal tissue healing processes can result in tissue fibrosis, which is characterized by excessive extracellular deposition of collagen and other extracellular matrix components. In the gastrointestinal (GI) tract, fibrosis typically starts with epithelial injury, such as in peptic injury of the distal esophagus or inflammatory bowel disease (IBD). Fibrosis in the GI tract may lead to narrowing of the lumen and stricture formation which may cause obstruction, surgery, and loss of bowel. There are no FDA-approved drugs to treat fibrosis in the GI tract and therefore, currently, the only treatment is surgical resection or endoscopic dilation. After treatment, however, many strictures recur, further leading to morbidity and mortality.

### SUMMARY

In some aspects, the presently disclosed subject matter provides a formulation comprising a plurality of sulconazole nanocrystals and one or more stabilizers.

In some aspects, the one or more stabilizers is selected from polyvinyl alcohol (PVA), hyaluronic acid (HA), carboxymethylcellulose (CMC), hydroxypropyl methylcellulose (HPMC), hydroxyethyl cellulose (HEC), sodium cholate (CHA), a cellulose derivative, a polysaccharide, polyethylene glycol, a poloxamer, and combinations thereof. In particular aspects, the poloxamer comprises poloxamer 407.

In some aspects, the concentration of sulconazole is between about 10 and about 500 mg/mL. In particular aspects, the formulation comprises: (a) between about 1.5% to about 5% PVA; (b) between about 0.5% and 1% HA; (c) between about 1% to about 2% CMC; (d) between about 1% HPMC to about 5% HPMC; and (e) between about 2% to about 6% poloxamer 407.

In some aspects, the formulation is lyophilized.

In some aspects, the presently disclosed subject matter provides a precursor formulation comprising the presently described formulation and a plurality of milling beads. In particular aspects, the plurality of milling beads comprise zirconium oxide beads. In more particular aspects, the formulation comprises about 500-mg sulconazole, about 2.0 g of 0.5-mm zirconium oxide beads, and about 1 mL of 2% (w/v) poloxamer 407.

In other aspects, the presently disclosed subject matter provides a method for treating or preventing fibrosis or intestinal re-stricturing in a gastrointestinal (GI) tract of a subject in need of treatment thereof, the method comprising administering to the subject a presently described formulation. In certain aspects, the administering of the formulation is via injection. In particular aspects, the injection comprises an intraperitoneal (IP) or a subcutaneous (SC) injection. In more particular aspects, the formulation is injected in a proximity of a stricture site. In yet more particular aspects, the formulation is injected in a proximity of a stricture site after a surgical or endoscopic procedure.

In certain aspects, the fibrosis is associated with an inflammatory bowel disease (IBD). In particular aspects, the inflammatory bowel disease is selected from Crohn's disease and (CD), ulcerative colitis (UC), and combinations thereof.

In certain aspects, the administration of the formulation modulates an acute healing response and/or interrupts one or more pathological fibrotic tissue remodeling processes. In certain aspects, the method comprises a decrease in a collagen layer thickness in a small intestine of the subject. In certain aspects, the administration of the formulation results in a folded, flexible epithelial structure more similar to healthy tissue.

In particular aspects, the administration of the formulation is a sustained-release administration.

In certain aspects, a concentration of sulconazole in the formulation has a range between about 100 mg/mL and about 500 mg/mL. In certain aspects, a volume of the formulation injected has a range between about 10 μL to about 100 μL. In certain aspects, the formulation is injected with a dose of sulconazole between about 100 mg/kg and about 1875 mg/kg.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, and FIG. 1C demonstrate that sulconazole inhibits the expression of key fibrotic genes in CCD-18Co and LX2 cell lines activated by TGFβ1. (FIG. 1A) CCD-18Co colon fibroblasts stimulated with TGF-β were left untreated (Untreated) or were treated with pirfenidone (4 mM) or sulconazole (10 μM) prior to staining for alpha smooth muscle actin (α-SMA, red), intracytoplasmic type 1 collagen (COL1A1, green), and cell nuclei (blue). (FIG. 1B) Western blot analysis displayed reduced expression of α-SMA and COL1A1 in stimulated CCD-18Co and LX2 cells treated with 10-μM suconazole compared to stimulated, untreated cells (Untreated). (FIG. 1C) RT-PCR demonstrated that sulconazole decreased expression of α-SMA (ACTA2) at much lower drug concentrations (1-10 μM) in CCD-18Co (n=3-5) and LX2 (n=3) cells compared to pirfenidone (4 mM). Dotted line represents normalization of control stimulated, untreated cells. Data presented as mean±SEM, *p<0.05 compared to untreated control cells;

FIG. 2A, FIG. 2B, FIG. 2C and FIG. 2D show sulconazole nanocrystal (sul-NC) characterization. (FIG. 2A) Transmission electron microscopy (TEM) image of 500-mg/mL Sul-NCs. Scale bar represents 500 nm. (FIG. 2B) Bar graph showing 500-mg/mL Sul-NC size over nine batches with the overall average (Ave.) across batches shown as mean±SD. (FIG. 2C) Sulconazole release under accelerated in vitro conditions in a rapid equilibrium dialysis device for Sul-NCs compared to free sulconazole. Data shown as mean±SD. (FIG. 2D) Stability of the 500-mg/mL Sul-NC as assessed by particle size during storage at room temperature or at 4° C. for 112 days. Data shown as mean±SD;

FIG. 3 is a scheme of a bleomycin-induced mouse skin fibrosis model. The mouse was shaved on the back in a small region and 5 spots were marked for injection. The bleomycin was injected every other day in different sectors, progressing from 1-5. Sul-NC (50 mg/kg or 150 mg/kg) were injected once weekly in sector 5;

FIG. 4A and FIG. 4B show Sul-NC reduced fibrotic collagen deposition and skin thickening in a bleomycin-induced mouse fibrosis model. Representative tissue sections from mice with bleomycin-induced skin fibrosis (n=5) after once weekly injection of Sul-NC (50 mg/kg or 150 mg/kg), IP injection of sulconazole (Free Sul, 10 mg/kg) every other day (Every 2d IP), or daily oral administration of pirfenidone (100 mg/kg). Mice not induced with bleomycin shown as Sham, and bleomycin with vehicle injections shown as Vehicle. (FIG. 4A) H&E staining of skin tissue sections showing the dermal thickness for each treatment group (representative dermis areas shown by double sided arrows). (FIG. 4B) Mason's trichrome staining of tissue sections shows the thickness and collagen layer of samples corresponding to each treatment group. Scale bar=100 μm;

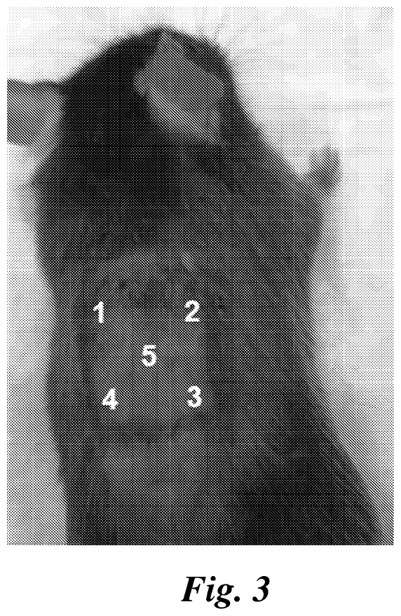

FIG. 3 shows once weekly injection of Sul-NCs reduced dermal thickening in a bleomycin-induced mouse fibrosis model. Quantification of dermal thickness from the bleomycin-induced skin fibrosis model (n=5) after once weekly injection of Sul-NC (50 mg/kg or 150 mg/kg), IP injection of sulconazole (Free Sul, 10 mg/kg) every other day (Every 2d IP), or daily oral administration of pirfenidone (100 mg/kg). Mice not induced with bleomycin shown as Sham, and bleomycin with vehicle injections shown as Vehicle. Data shown as mean±SD, * p<0.01 compared to Vehicle;

FIG. 6 shows experimental procedures to create a small intestine fibrosis mouse model to test the efficacy of Sul-NC for the prevention of fibrosis. A 6-mm section of intestine is resected from one C57BL/6 mouse, and cut into equal 1 mm in length pieces. Each piece is then transplanted under the skin in the neck in another mouse. The implants were excised on day 7 post-transplantation for assessment of fibrosis;

FIG. 7A, FIG. 7B, FIG. 7C, FIG. 7D, and FIG. 7E demonstrate that Sul-NC reduces collagen deposition in a mouse model of intestinal tissue fibrosis. Representative Masson's Trichrome stained intestinal tissue sections from mice (n=3-5) treated with (FIG. 7A) PBS (Vehicle), (FIG. 7B) a single injection of Sul-NC at 50 mg/kg, (FIG. 7C) a single injection of Sul-NC at 150 mg/kg or (FIG. 7D) three times daily oral gavage with pirfenidone at 100 mg/kg for 7 days (yellow arrows note collagen capsule). (FIG. 7E) The collagen layer thickness in small intestine grafts was significantly decreased with the single Sul-NC injections and the daily oral pirfenidone treatment. Data shown as mean±SEM, *p<0.01 compared to untreated Control;

FIG. 8A, FIG. 8B, and FIG. 8C demonstrate that Sul-NC is effective in preventing fibrosis in pig esophagus stricture model. (FIG. 8A) Representative endoscopic images of the esophageal lumen at the stricture sites (30, 40, and 50 cm from the incisors) in pigs treated with Vehicle (2% F127) or Sul-NC. (FIG. 8B) X-ray images of strictures after treatment with Vehicle or Sul-NC (yellow arrows point to esophageal lumen). (FIG. 8C) The luminal diameter was measured from tissue sections from pigs treated with Vehicle (n=12 strictures from 5 pigs) or Sul-NC (n=9 strictures from 5 pigs). Data shown as mean±SEM. *p<0.05 compared to Vehicle;

FIG. 9 demonstrates that Sul-NC is effective in preventing fibrosis in pig esophagus stricture model. Representative images of the esophageal lumen from pigs treated with Vehicle or Sul-NC at the site of the stricture compared to healthy tissue sections from the uninvolved regions of the esophagus. Scale bar represents 5 mm;

FIG. 10A, FIG. 10B, and FIG. 10C illustrate an experiment in which Sul-NC were formulated at 500 mg/mL in 2% F127 and stored at either room temperature (RT), at 4° C., or were lyophilized with no cryoprotectant and stored at RT. Sul-NC (FIG. 10A) particle size, (FIG. 10B) PDI, and (FIG. 10C) zeta potential were measured over 168 days. The native formulation showed good stability under storage at RT and 4° C. Data shown as mean±SD;

FIG. 11A, FIG. 11B, and FIG. 11C illustrate an experiment in which Sul-NC were formulated at 500 mg/mL in 2% w/w F127 and then left undiluted or diluted 1:10 in either 2% F127 or water prior to lyophilization. The lyophilized powders were then reconstituted in water and characterized. Measurements of (FIG. 11A) particle size, (FIG. 11B) PDI, and (FIG. 11C) zeta potential were taken after reconstitution in water. Dilution prior to lyophilization did not appear to improve particle properties after reconstitution. Data shown as mean±SD. 1: Non-diluted, lyophilized; 2: Diluted 1:10 in 2% F127, lyophilized; 3: Diluted 1:10 in water, lyophilized;

FIG. 12A and FIG. 12B illustrate an experiment in which Sul-NC were formulated at 25 mg/mL in 0.25%, 0.5%, or 1% (w/v) hyaluronic acid (HA) and stored at 4° C. Sul-NC (FIG. 12A) particle size and (FIG. 12B) zeta potential were measured over 8 days. Sul-NC formulated with HA were larger than those formulated with 2% F127, possibly due to the larger molecular weight and increased viscosity of hyaluronic acid. Sul-NC formulated with 0.5% hyaluronic acid showed good stability in size over time. Particle zeta potential was increasingly negative with increasing hyaluronic acid concentration due to the polyanionic nature of the polymer. Data shown as mean SD;

FIG. 13A and FIG. 13B illustrate an experiment in which Sul-NC were formulated at 50 mg/mL, 100 mg/mL, or 200 mg/mL sulconazole concentration in 5% (w/v) hydroxypropyl methylcellulose (HPMC) and stored at 4° C. Sul-NC (FIG. 13A) particle size and (FIG. 13B) zeta potential were measured over 21 days. HPMC coating at lower sulconazole concentrations appeared to create stable particles with a near neutral zeta potential. Data shown as mean±SD; and

FIG. 14A and FIG. 14B illustrate an experiment in which Sul-NC were formulated with 50 mg/mL, 100 mg/mL, or 200 mg/mL in 5% (w/v) poly(vinyl alcohol) (PVA) and stored at 4° C. Sul-NC (FIG. 14A) particle size and (FIG. 14B) zeta potential were measured over 21 days. Similar to HPMC, 50 mg/mL and 100 mg/mL Sul-NC with a PVA coating appeared stable with near neutral zeta potential. Overall, the particle sizes were larger than when formulating with 2% F127. Data shown as mean±SD.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides a formulation comprising a plurality of sulconazole nanocrystals and one or more stabilizers.

In certain embodiments, the particle size of the sulconazole nanocrystals has a range from about 100 to about 600 nm, including about 100, 150, 200, 250, 300, 350, 400, 450, 500, 550, 600 nm including any whole or fractional integer in between. In particular embodiments, the particle size of the sulconazole nanocrystals is about 200 nm.

In certain embodiments, the one or more stabilizers is selected from polyvinyl alcohol (PVA), hyaluronic acid (HA), carboxymethylcellulose (CMC), hydroxypropyl methylcellulose (HPMC), hydroxyethyl cellulose (HEC), sodium cholate (CHA), a poloxamer, and combinations thereof. As used herein, the term “poloxamer” refers to nonionic triblock copolymers comprising a central hydrophobic chain of polyoxypropylene (i.e., poly(propylene oxide)) flanked by two hydrophilic chains of polyoxyethylene (i.e., poly(ethylene oxide)), i.e., poly(ethylene oxide)-b-poly(propylene oxide)-b-poly(ethylene oxide) (PEO-PPO-PEO).

Poloxamers have the general structure of:

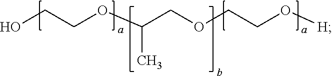

wherein each a is an integer from about 2 to about 130 and b is an integer from about 15 to about 67. Poloxamers also are also known under the trade names Pluronic®, Synperonic™. Kolliphor®, and Lutrol® F. Because of the variability in each “a” and “b” in the poloxamer chemical structure provided hereinabove, many different poloxamers exist, with molecular weights and ethylene oxide-propylene oxide weight ratios varying from 1100 to 14,000 and 1:9 to 8:2, respectively, each of which can have different properties.

Poloxamers are typically named starting with the letter “P” (for poloxamer) followed by three digits, where the first two digits multiplied by 100 give the approximate molecular mass of the polyoxypropylene core, and the last digit multiplied by 10 gives the percentage polyoxyethylene content. For example, a poloxamer referred to as “P407” would have a polyoxypropylene molecular mass of 4000 g/mol and 70% polyoxyethylene content.

Further, the naming convention for commercial embodiments, such as the Pluronic® tradename, starts with a letter defining its physical form at room temperature (L=liquid, P=paste, and F=flake (solid), followed by two or three digits. The first digit (two digits in a three-digit number) in the numerical designation, multiplied by 300, indicates the approximate molecular weight of the polyoxypropylene core, and the last digit multiplied by 10 gives the percentage polyoxyethylene content. For example, Pluronic® F127 is a flake (or solid) poloxamer having an approximate weight of the polyoxypropylene core of approximately 3600 and 70% polyoxyethylene content. Pluronic® F127 also is referred to as Synperonic™ PE/F-127, Kolliphor® P 407, and poloxamer 407. In particular embodiments, the poloxamer is Pluronic® F127 (poloxamer 407).

Other suitable poloxamers include those in the Pluronic® family including, but not limited to Pluronic® P84, P85, F88, F98, F108, P102, P103, P104, P105, P123, and F108.

In certain embodiments, the concentration of sulconazole is between about 10 and about 500 mg/mL. In particular embodiments, the formulation comprises: (a) between about 1.5% to about 5% PVA; (b) between about 0.5% and 1% HA; (c) between about 1% to about 2% CMC; (d) between about 1% HPMC to about 5% HPMC; and (e) between about 2% to about 6% poloxamer 407.

In certain embodiments, the formulation is lyophilized. In certain embodiments, the lyophilized formulation further comprises a cryoprotectant. In other embodiments, the lyophilized formulation does not comprise a cryoprotectant.

In other embodiments, the presently disclosed subject matter provides a precursor formulation comprising the formulation described immediately hereinabove and a plurality of milling beads. In particular embodiments, the plurality of milling beads comprise zirconium oxide beads. The zirconium oxide beads can be used to homogenize the formulation during mixing and can then be removed from the formulation by filtering prior to injection. In yet more particular embodiments, the formulation comprises about 500-mg sulconazole, about 2.0 g of 0.5-mm zirconium oxide beads, and about 1 mL of 2% (w/v) poloxamer 407.

In other embodiments, the presently disclosed subject matter provides a method for treating or preventing fibrosis or intestinal re-stricturing in a gastrointestinal (GI) tract of a subject in need of treatment thereof, the method comprising administering to the subject a formulation described hereinabove. As used herein, the term “stricture” refers to an abnormal narrowing of a bodily passage, as from inflammation, cancer, or the formation of scar tissue. In some embodiments, the stricture is in an intestine of a subject. In other embodiments, the stricture is in the colon of the subject. In other embodiments, the stricture is in the esophagus of the subject.

In certain embodiments, the administering of the formulation is via injection. In particular embodiments, the injection comprises an intraperitoneal (IP) or a subcutaneous (SC) injection.

In certain embodiments, the formulation is injected in a proximity of a stricture site. In particular embodiments, the formulation is injected in a proximity of a stricture site after a surgical or endoscopic procedure. As used herein, the term “proximity” refers to a location about 2 cm from the stricture site, including about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0 cm.

In certain embodiments, the fibrosis is associated with an inflammatory bowel disease (IBD). In particular embodiments, the inflammatory bowel disease is selected from Crohn's disease and (CD), ulcerative colitis (UC), and combinations thereof.

In certain embodiments, the formulation modulates an acute healing response and/or interrupts one or more pathological fibrotic tissue remodeling processes. In certain embodiments, the method comprises a decrease in a collagen layer thickness in a small intestine of the subject. In certain embodiments, the administration of the formulation results in a folded, flexible epithelial structure more similar to healthy tissue.

In particular embodiments, the administration of the formulation is a sustained-release administration, such that drug is released for more than 3 days in vivo, including more than 3 days, 7 days, 30 days, 60 days, and 90 days.

In certain embodiments, a concentration of sulconazole in the formulation has a range between about 100 mg/mL and about 500 mg/mL. In certain embodiments, a volume of the formulation injected has a range between about 10 μL to about 100 L. In certain embodiments, the formulation is injected with a dose of sulconazole between about 100 mg/kg and about 1875 mg/kg.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the drug target, and the like.

The term “combination” is used in its broadest sense and means that a subject is administered at least two agents, more particularly the presently disclosed formulation in combination with a second therapeutic agent or therapy. More particularly, the term “in combination” refers to the concomitant administration of two (or more) active agents or therapies for the treatment of a single disease state. As used herein, the active agents or therapies may be combined and administered in a single dosage form, may be administered as separate dosage forms at the same time, or may be administered as separate dosage forms that are administered alternately or sequentially on the same or separate days. In one embodiment of the presently disclosed subject matter, the active agents or therapies are combined and administered in a single dosage form. In another embodiment, the active agents or therapies are administered in separate dosage forms (e.g., wherein it is desirable to vary the amount of one but not the other). The single dosage form may include additional active agents therapies for the treatment of the disease state.

Further, the presently disclosed formulation in combination an additional therapeutic agent or therapy can be further administered with adjuvants that enhance stability of the agents, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the presently disclosed formulation in combination with an additional therapeutic agent or therapy can be varied so long as the beneficial effects of the combination of these agents are achieved. Accordingly, the phrase “in combination with” refers to the administration of the presently disclosed formulation described herein and an additional therapeutic agent or therapy either simultaneously, sequentially, or a combination thereof. Therefore, a subject administered a combination of a presently disclosed formulation and an additional therapeutic agent or therapy can receive the presently disclosed formulation and additional therapeutic agent or therapy at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed formulation and additional agent or therapy are administered simultaneously, they can be administered to the subject as separate pharmaceutical compositions, each comprising either the presently disclosed formulation or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + B / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### An Injectable Sustained-Release Antifibrotic Nanoformulation for Preventing Fibrosis and Intestinal Re-Stricturing

### 1.1 Overview

The presently disclosed subject matter includes injectable formulations for providing sustained delivery of antifibrotic drugs at the stricture site to prevent recurrence after surgical or endoscopic treatment. A small molecule drug library screen uncovered strong antifibrotic properties for sulconazole, an FDA-approved drug for treating fungal infections. Since free sulconazole has a short life that is insufficient to treat fibrosis during the wound healing process, sulconazole nanocrystals (Sul-NC) were formulated for sustained release. Importantly, it was found that Sul-NC dramatically increased the maximum tolerated dose in mice, and provided superior or equivalent fibrosis prevention with less frequent dosing in mouse models of skin and intestinal tissue fibrosis. In a swine model of esophageal stricture, a single injection of Sul-NC after balloon dilation prevented re-stricturing as measured by a significant increase in the esophageal diameter compared to placebo.

### 1.2 Background

Normal tissue repair involves immune, as well as mesenchymal cell activation, extracellular matrix (ECM) deposition, and tissue remodeling. Rieder et al., 2007. Severe or repetitive injury, such as in the case of chronic inflammation, however, can lead to excessive ECM accumulation and the formation of fibrotic tissue. Rieder et al., 2007. Fibrosis can affect any organ, and can lead to disruption of normal tissue structure and organ dysfunction or failure. Henderson et al., 2020. Myofibroblasts display a high expression of α-smooth muscle actin (α-SMA), are capable of contractility, and play central roles in fibrosis and scar formation. Rieder et al., 2007. Chronic inflammation causes impaired mucosal tissue repair and myofibroblast proliferation. Rieder, 2013; Rieder and Fiocchi, 2009. Hence, targeting the myofibroblast proliferation pathway has been proposed as a strategy for the development of effective antifibrotic drugs. Henderson et al., 2020; Bollong et al., 2017; Rosenbloom et al., 2013. To date, there are only 2 FDA-approved antifibrotic drugs in general, pirfenidone and nintedanib, both for the treatment of idiopathic pulmonary fibrosis. Raghu and Selman, 2015. To date, however, there are no antifibrotic drugs for any other indication, including intestinal fibrosis. Thus, effective antifibrotic therapies represent a critically important unmet need.

In the gastrointestinal (GI) tract, inflammatory bowel disease (IBD) is the main cause of intestinal fibrosis and exemplifies the significant morbidity deriving from intestinal fibrosis. Latella and Rieder, 2017. IBD can be separated into two major subtypes, Crohn's disease (CD) and ulcerative colitis (UC), and affects more than three million people in the United States. Dahlhamer et al., 2015. Fibrosis is the underlying mechanism for the development of intestinal strictures, Rieder et al., 2007; Schmoyer et al., 2021, a narrowing of the intestine, which occur in 27%-54% of Crohn's patients and 1.5%-11.2% of UC patients. Le Berre et al., 2020. Such strictures often require surgical or endoscopic treatment, and almost invariably recur due to ongoing fibrosis. Rieder et al., 2007. Recurrent fibrotic strictures typically lead to multiple surgeries, short gut, stomas, poor quality of life, and high cost of healthcare. Lan et al., 2018. Since pro-fibrotic mechanisms in IBD are triggered by inflammation, there was significant hope that potent anti-inflammatory therapies developed over the past 20 years would decrease the incidence of stricturing/fibrosing complications. This expectation, however, did not come to fruition. Cosnes et al., 2005; D'Amico et al., 2020; Li et al., 2019; Moschen et al., 2019. A dedicated antifibrotic treatment, developed and formulated for the needs of fibrotic strictures in the GI tract appears urgently needed.

Intestinal strictures offer the opportunity for administering localized, targeted therapy that would reduce the likelihood of systemic complications. Without wishing to be bound to any one particular theory, it is thought that local injection of antifibrotic drugs at the time of endoscopic dilation has the potential for modulating wound healing and fibrotic processes with reduced risk of systemic side effects. Of note, prior efforts involving single injections of drugs, such as corticosteroids and mitomycin C, were largely ineffective, Madadi-Sanjani et al., 2018; East et al., 2007, likely in part due to the short duration of action before being cleared from the body. Without wishing to be bound to any one particular theory, it is thought that a sustained-release formulation is optimal for positively impacting tissue remodeling processes, such as fibrosis, which occur over days and weeks.

The presently disclosed subject matter demonstrates that sulconazole, a topical antifungal agent, has potent antifibrotic activity. Again, without wishing to be bound to any one particular theory, it is thought that providing sustained, effective concentrations of sulconazole locally at the tissue site could modulate the acute healing response and interrupt pathological fibrotic tissue remodeling processes. The presently disclosed subject matter demonstrates that a sulconazole nanocrystal (Sul-NC) formulation was highly effective in preventing fibrosis in rodent models of skin and intestinal fibrosis, as well as a novel swine model of esophageal stricture. Li et al., 2021. Finally, the presently disclosed subject matter demonstrates that Sul-NC was well-tolerated and safe. The presently disclosed subject matter data build a foundation to support further preclinical studies leading to the development of Sul-NC as an antifibrotic drug.

### 1.3 Results

### 1.3.1 High Throughput Microscope Based Screen Identified Sulconazole as an Antifibrotic Agent

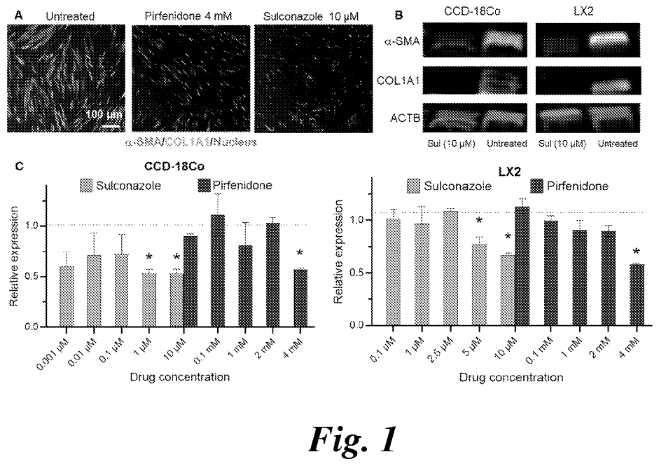

To identify potential antifibrotic agents, a high-throughput microscope-based drug screening approach using human primary colonic fibroblasts (CCD-18Co) activated with transforming growth factor-β (TGF-β) was used. A panel of 1,586 FDA-approved small molecule drugs were then screened for their ability to reduce expression of α-SMA and type 1 collagen (COL1A1) as assessed by fluorescent immunocytochemistry. Based on its known antifibrotic effects, pirfenidone was included in this screen and found effective at a dose of 4 mM (FIG. 1A). Notably, sulconazole provided a similar decrease in the staining of α-SMA and COL1A1 at a much lower dose of 10 μM (FIG. 1A). Both activated CCD-18Co cells and human hepatic stellate cells (LX2) were then used in a confirmatory Western blot analysis, that showed sulconazole significantly reduced the protein production of α-SMA and COL1A1 (FIG. 1B). α-SMA expression was then analyzed by RT-PCR in both activated CCD-18Co cells and LX2 cells. Sulconazole at concentrations as low as 1 μM and 5 μM significantly reduced ACTA2 expression in activated CCD-18Co and LX-2 cells, respectively, whereas pirfenidone showed no effect until reaching 4-mM concentration (FIG. 1C). These in vitro results indicated that sulconazole is a potent antifibrotic candidate, and acts at much lower concentration than that of pirfenidone.

### 1.3.2 Sulconazole can be Formulated for Sustained Release

An array of generally regarded as safe (GRAS) stabilizers was tested at a range of concentrations for use in nanomilling of sulconazole. The resulting nanocrystals varied in size and polydispersity, and generally larger molecular weight polymer stabilizers, such as hyaluronic acid (HA), resulted in larger and more polydisperse particle sizes (Table 1).

TABLE 1
Sulconazole particle size, polydispersity index (PDI),
and ζ-potential when formulated at a range of drug
concentrations (10-200 mg/mL) and with 0.5-5% polyvinyl
alcohol (PVA), hyaluronic acid (HA), carboxymethyl cellulose
(CMC), and hydroxypropyl methylcellulose (HPMC).

| Sulco- | | | | |
|---|---|---|---|---|
| nazole | Stabilizer | Diameter | | ζ-potential |
| (mg/mL) | (w/v) | (nm) | PDI | (mV) |
| 10 | 1.5% PVA | 271 ± 16 | NR | NR |
| 10 | 2% PVA | 296 ± 25 | NR | NR |
| 10 | 0.5% HA | 1010 ± 96 | NR | NR |
| 10 | 1% HA | 2500 ± 45 | NR | NR |
| 10 | 1% CMC | 714 ± 12 | NR | NR |
| 10 | 2% CMC | 970 ± 84 | NR | NR |
| 25 | 1% PVA | 338 ± 17 | 0.48 ± 0.02 | −0.43 ± 0.05 |
| 25 | 2% PVA | 229 ± 52 | 0.82 ± 0.16 | −1.8 ± 0.25 |
| 25 | 5% PVA | 206 ± 43 | 0.97 ± 0.05 | −1.1 ± 0.30 |
| 25 | 0.25% HA | 1726 ± 121 | 1.0 ± 0.00 | −5.9 ± 0.83 |
| 25 | 0.5% HA | 6190 ± 1457 | 0.87 ± 0.21 | −11.9 ± 4.9 |
| 25 | 1% HA | 5817 ± 1181 | 0.57 ± 0.48 | −9.4 ± 6.3 |
| 25 | 1% HPMC | 656 ± 93 | 0.59 ± 0.43 | −0.64 ± 1.7 |
| 25 | 2% HPMC | 268 ± 2 | 0.52 ± 0.14 | −11.1 ± 1.7 |
| 25 | 5% HPMC | 319 ± 263 | 0.92 ± 0.17 | −2.3 ± 1.2 |
| 50 | 5% PVA | 476 ± 3 | 0.06 ± 0.04 | −1.0 ± 0.49 |
| 50 | 5% HPMC | 348 ± 24 | 0.13 ± 0.05 | −0.80 ± 1.6 |
| 100 | 5% PVA | 379 ± 15 | 0.09 ± 0.06 | −0.43 ± 1.2 |
| 100 | 5% HPMC | 375 ± 10 | 0.12 ± 0.10 | 0.72 ± 0.30 |
| 200 | 5% PVA | 422 ± 15 | 0.13 ± 0.08 | −0.03 ± 0.52 |
| 200 | 5% HPMC | 359 ± 41 | 0.10 ± 0.06 | 1.9 ± 0.44 |

Data presented as mean ± SD.
NR = Not recorded

Incorporation of Pluronic F127 generally led to smaller and more uniform particle sizes, even as the concentration of sulconazole was increased from 10 mg/mL to 500 mg/mL (see Table 2).

TABLE 2
Sul-NC particle size, polydispersity index (PDI),
and ζ-potential when formulated at a range of
drug concentrations (10-500 mg/mL) and with 2-6% F127.

| Sulconazole | F127 | Diameter | | ζ-potential |
|---|---|---|---|---|
| (mg/mL) | (w/v) | (nm) | PDI | (mV) |
| 10 | 2% | 242 ± 6.1 | 0.11 ± 0.06 | 1.5 ± 0.16 |
| 30 | 2% | 247 ± 4.5 | 0.10 ± 0.02 | 2.1 ± 0.50 |
| 30 | 4% | 255 ± 5.1 | 0.07 ± 0.05 | 1.7 ± 0.29 |
| 30 | 6% | 290 ± 1.6 | 0.13 ± 0.02 | 1.4 ± 0.64 |
| 50 | 2% | 226 ± 7.7 | 0.20 ± 0.01 | 1.9 ± 0.12 |
| 50 | 4% | 250 ± 6.3 | 0.15 ± 0.02 | 1.8 ± 0.21 |
| 50 | 6% | 261 ± 7.5 | 0.16 ± 0.01 | 1.8 ± 0.23 |
| 75 | 2% | 217 ± 12 | 0.17 ± 0.10 | 2.4 ± 0.09 |
| 75 | 4% | 233 ± 4.2 | 0.11 ± 0.05 | 2.3 ± 0.18 |
| 75 | 6% | 248 ± 8.0 | 0.14 ± 0.06 | 2.1 ± 0.26 |
| 100 | 2% | 215 ± 7.7 | 0.18 ± 0.06 | 2.4 ± 0.16 |
| 100 | 4% | 215 ± 11 | 0.22 ± 0.01 | 2.4 ± 0.21 |
| 100 | 6% | 223 ± 11 | 0.23 ± 0.03 | 2.3 ± 0.14 |
| 200 | 2% | 185 ± 8.4 | 0.27 ± 0.02 | 2.7 ± 0.36 |
| 500 | 2% | 232 ± 19 | 0.35 ± 0.06 | −0.31 ± 0.87 |

Data presented as mean ± SD.

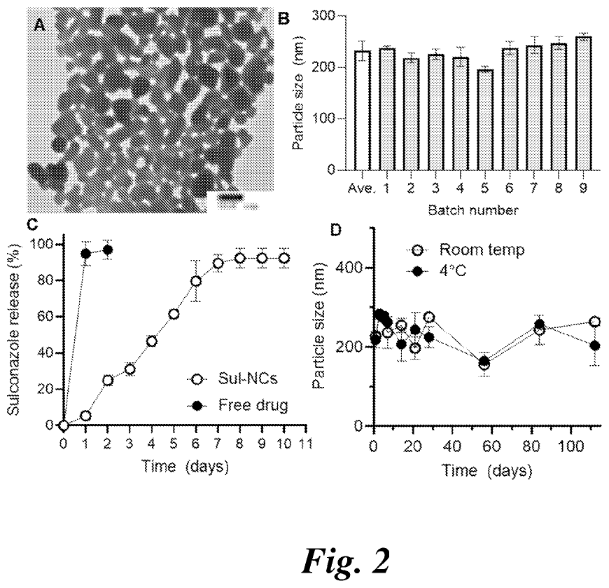

While 2-6% Pluronic F127 resulted in similar particle sizes, 2% was chosen in the final formulation due to decreased viscosity, which is preferred for injection through an endoscopy needle. Electron microscopy of the 500 mg/mL Sul-NC showed non-spherical particles with cuboidal edges, reflective of the crystalline nature of the drug (FIG. 2A). The particle size was consistent across nine batches produced on different days, ranging in size from 196-261 nm (average across batches 232±19 nm) (FIG. 2B). Using a rapid equilibrium dialysis system for in vitro drug release under accelerated conditions, that free sulconazole was not impeded by the dialysis membrane and that the Sul-NC dissolved over a period of 7-8 days was confirmed (FIG. 2C). The 500-mg/mL Sul-NC also was stable over 112 days of storage at room temperature or at 4° C., as assessed by measuring the particle size (FIG. 2D).

### 1.3.3 Sul-NC Increased the Sulconazole Maximum Tolerated Dose (MTD) In Vivo

To evaluate the safety of Sul-NC, as well as inform the dose for efficacy experiments, the maximum tolerated dose (MTD) was determined free sulconazole and Sul-NC. First, three groups of 5 mice each received an intraperitoneal (IP) injection with a single dose of 15 mg/kg, 30 mg/kg, or 40 mg/kg free sulconazole. All mice survived (Table 3). Increasing the IP dose to 50 mg/kg, however, resulted in a survival rate of only 3 of 9 mice (33%) (Table 3). Similarly, dosing sulconazole at 50 mg/kg via the subcutaneous (SC) route resulted in survival of only 2 of 4 mice (50%) (Table 3). All mice that did not survive died within 1-4 days of injection (not shown).

TABLE 3
Determination of maximum tolerated dose of free sulconazole injected
intraperitoneally (IP) or subcutaneously (SC) at different doses.
Mice that did not survive died within 1-4 days of injection.

| Dose (mg/kg) | Route | Survival |
|---|---|---|
| 15 | IP | 5/5 (100%) |
| 30 | IP | 5/5 (100%) |
| 40 | IP | 5/5 (100%) |
| 50 | IP | 3/9 (33%) |
| 50 | SC | 2/4 (50%) |

The effect of nanoformulation on the tolerability of sulconazole injected SC was then investigated. Having previously observed that injection volume can affect the rate and extent of drug uptake from crystalline formulations, Hsueh et al., 2021, different injection volumes (10-100 μL) and Sul-NC concentrations (100-500 mg/mL) also were tested. It was found that a combination of higher injection volume (50-100 μL) and lower sulconazole concentration (100-200 mg/mL) showed higher mortality (Table 4). For example, only 2 of 3 (66%) of mice survived after subcutaneous injection of 100 L of the 100 mg/mL formulation (500 mg/kg), whereas there was 100% survival when injecting 50 μL of the 500 mg/mL formulation (1250 mg/kg) (Table 4). This observation suggested that increasing the formulation concentration and using a smaller injection volume aided in slowing the rate of systemic drug absorption. As the Sul-NC dose was increased to 1,875 mg/kg, however, only 2 out of 4 mice survived (50%), and at 2,500 mg/kg, 0 out of 4 mice survived (0%). It is possible that a smaller injection volume (<75 μL) would have resulted in improved tolerability, but the concentration was the limiting factor at such high doses. Overall, Sul-NC doses as high as 1,250 mg/kg were well-tolerated, whereas a free sulconazole dose of only 50 mg/kg resulted in reduced survival.

TABLE 4
Determination of maximum tolerated dose of Sul-NC
injected subcutaneously (SC) at different concentrations
and injection volumes. Mice that did not survive
died within 1-4 days of injection.

| Concentration | Volume | Dose | |
|---|---|---|---|
| (mg/mL) | (μL) | (mg/kg) | Survival |
| 100 | 50 | 250 | 4/4 (100%) |
| 100 | 100 | 500 | 2/3 (66%) |
| 200 | 10 | 100 | 3/3 (100%) |
| 200 | 25 | 250 | 3/3 (100%) |
| 200 | 50 | 500 | 2/3 (66%) |
| 200 | 100 | 1,000 | 1/3 (33%) |
| 300 | 10 | 150 | 5/5 (100%) |
| 300 | 25 | 375 | 3/3 (100%) |
| 400 | 10 | 200 | 3/3 (100%) |
| 400 | 25 | 500 | 3/3 (100%) |
| 500 | 10 | 250 | 4/4 (100%) |
| 500 | 25 | 625 | 4/4 (100%) |
| 500 | 30 | 750 | 3/3 (100%) |
| 500 | 40 | 1,000 | 3/3 (100%) |
| 500 | 50 | 1,250 | 3/3 (100%) |
| 500 | 75 | 1,875 | 2/4 (50%) |
| 500 | 100 | 2,500 | 0/4 (0%) |

To further investigate systemic drug toxicity, free sulconazole or Sul-NC was injected prior to collecting blood samples to evaluate markers of liver and kidney function. Using the 500 mg/mL Sul-NC, a single subcutaneous dose of 312.5 mg/kg, 625 mg/kg, or 1,250 mg/kg, respectively, was administered in mice. That the plasma concentrations of alanine aminotransferase (ALT), aspartate aminotransferase (AST), blood urea nitrogen (BUN), and creatine remained in the normal range at 6 h for all mice injected with Sul-NC was confirmed (Table 5). A single subcutaneous injection of the free drug at 50 mg/kg, however, increased the blood level of ALT and BUN, suggesting liver and kidney damage (Table 5). This difference in tolerated dose is reflective of the sustained-release properties of the nanocrystals, and suggested that the Sul-NC formulation was safe to use at high concentrations.

### 1.3.4 Sul-NC is an Effective Antifibrotic in Rodent Skin and Intestine Fibrosis Models

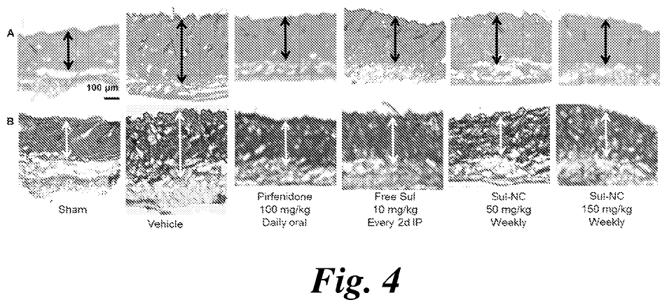

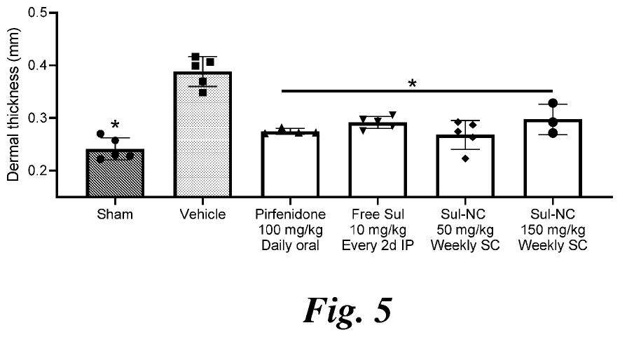

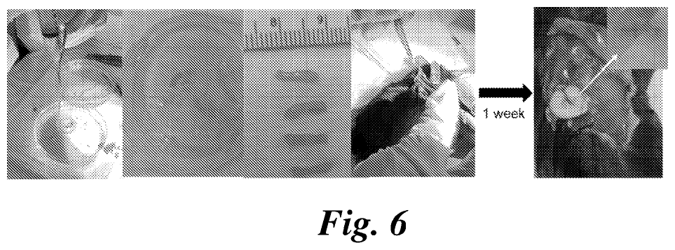

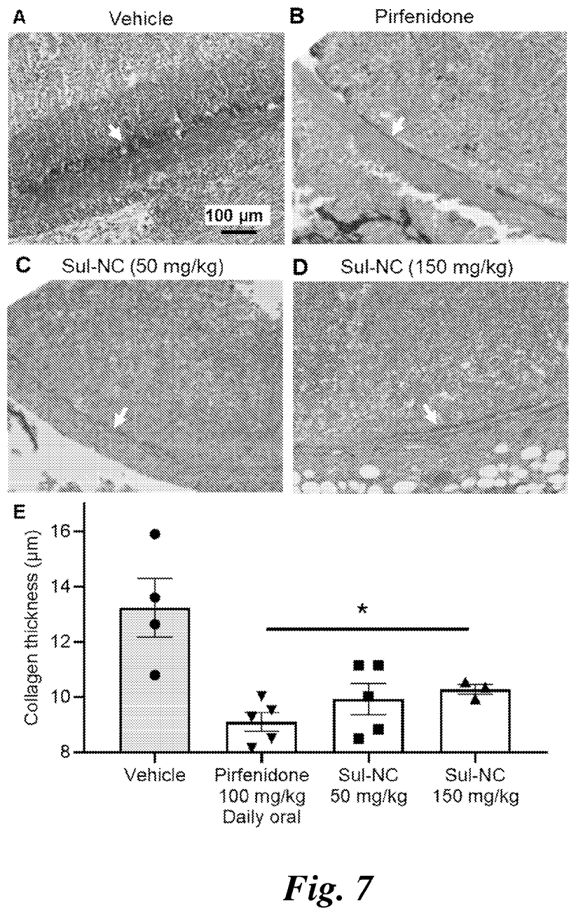

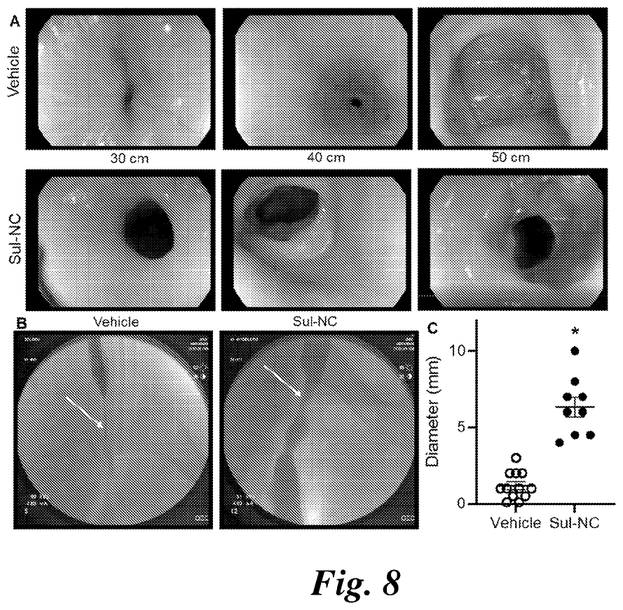

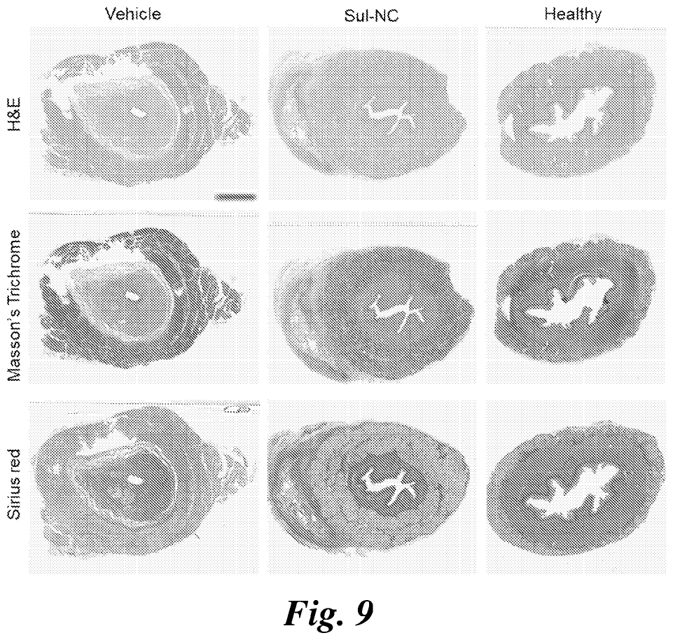

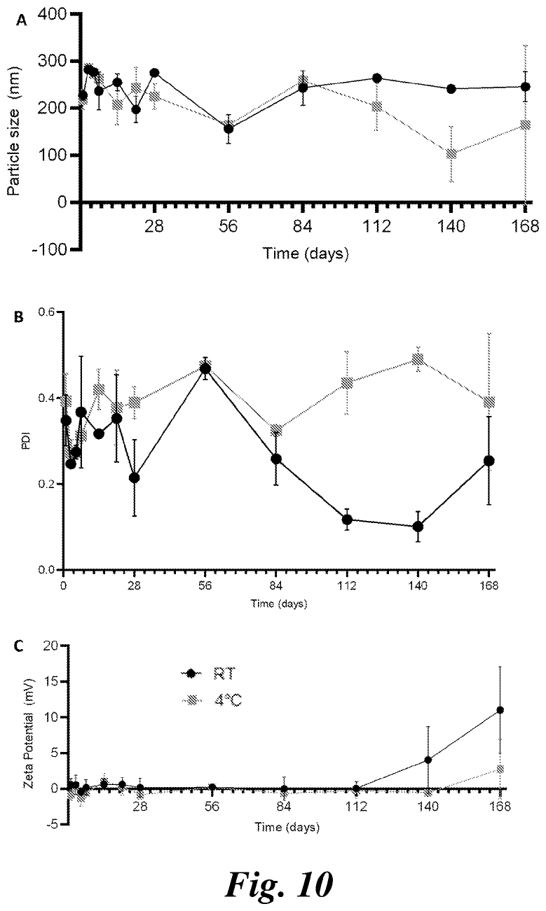

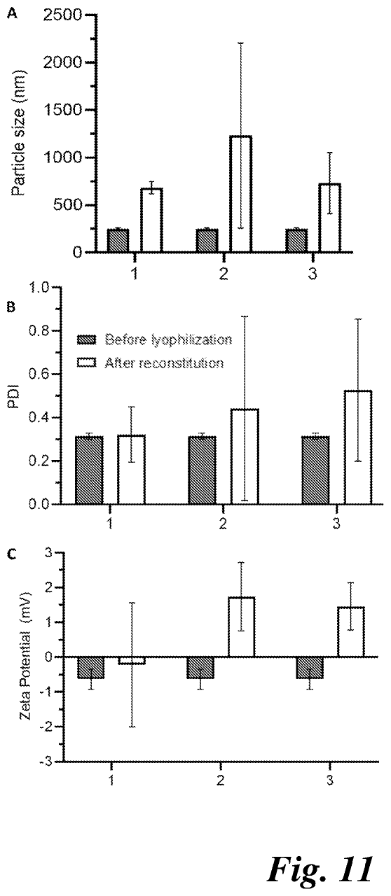

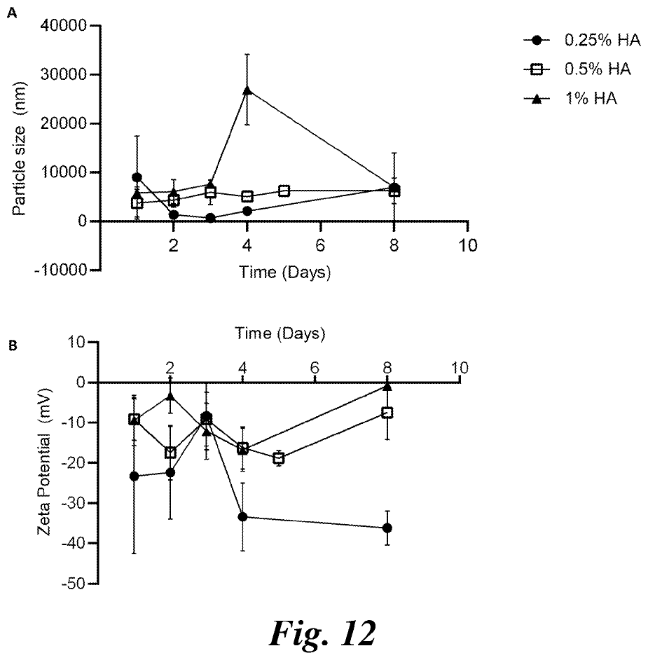

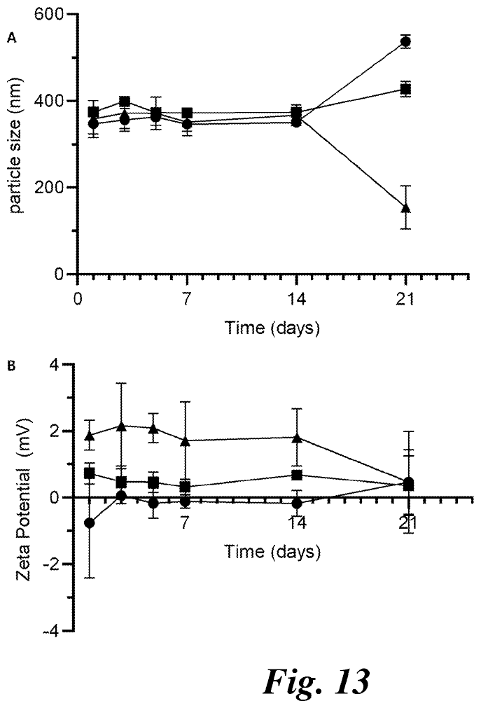

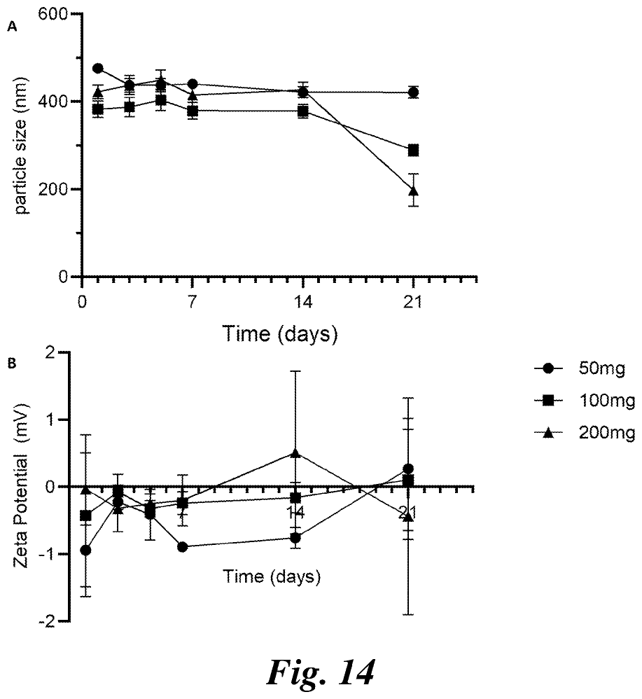

There is a paucity of animal models of GI tract fibrosis/strictures. Li et al., 2021. To obtain preliminary evidence regarding efficacy of Sul-NC as antifibrotic in vivo, a well-described and reproducible bleomycin-induced rodent skin fibrosis model was used. Yamamoto et al., 1999. To induce fibrosis, bleomycin was injected in mouse skin at five spots within a 1×1 cm region on the back every other day for four weeks (FIG. 3), as previously described. Yamamoto et al., 1999. Five groups of mice (n=5 each) were treated with either free sulconazole (Free Sul) at 10-mg/kg IP every other day, Sul-NC at 50-mg/kg or 150-mg/kg SC once per week, PBS (Vehicle) SC once per week, or daily oral pirfenidone at 100 mg/kg. Examining tissue sections of the skin samples dissected from regions injected with bleomycin by H&E staining or Masson's Trichrome staining (FIG. 4), it was found that all treatments significantly reduced the thickness of the dermis compared to Vehicle (FIG. 5). In addition, the Masson's Trichrome staining confirmed that the thickness of the dermis was due to deposition of collagen and other ECM proteins (blue staining in the figure). Of note, there was a striking difference between the dose and frequency of administration when comparing pirfenidone (daily dosing, total dose 2,800 mg/kg) vs Sul-NC (once weekly, total dose 200 mg/kg).

An intestine transplantation model (FIG. 6) that was previously used to validate the antifibrotic efficacy of oral pirfenidone was employed. Meier et al., 2016. Sul-NC at a dose of 50 mg/kg or 150 mg/kg was given as a single injection at the site of the transplant and compared to oral pirfenidone at the dose previously described to be effective in the model, three times daily oral dosing with 100 mg/kg. Tissue sections stained by Mason's Trichrome showed that Sul-NC significantly reduced collagen deposition (collagen layer thickness) at both 50 mg/kg (9.9±0.6 μm) and 150 mg/kg (10.3±0.2 μm) compared to Vehicle injected animals (13.2±1.1 μm), and was similar to the three times daily oral pirfenidone (9.1±0.3 μm) (FIG. 7). Again, there was a striking difference between the dose and frequency of administration when comparing pirfenidone (three times daily dosing, total dose 2,100 mg/kg) vs Sul-NC (single dose, 50 mg/kg).

### 1.3.5 Sul-NC is Effective in a Swine Model of Luminal GI Tract Strictures

A clinically relevant pig esophagus stricture model that includes endoscopic balloon dilation and subsequent stricture reformation was recently established. Li et al., 2021. Two weeks after induction, strictures with greater than 6-mm diameter were first endoscopically balloon dilated to 10 mm and then injected with either vehicle or Sul-NC. Two weeks post-treatment, endoscopy (FIG. 8A) and x-ray with contrast (FIG. 8B) were used to examine the stricture sites, and observed significantly larger luminal openings in the pigs treated with Sul-NC (6.3±0.5 mm) compared to Vehicle (1.2±0.5 mm) (FIG. 8C). Tissue sections stained with Masson's Trichrome and Sirius red showed significant reduction of collagen deposition in the sites injected with Sul-NC compared to Vehicle controls (FIG. 9). Further, the Sul-NC treated tissues showed a folded, flexible epithelial structure more similar to healthy tissue from uninvolved regions or the esophagus (FIG. 9). These results confirmed the antifibrotic effect of the Sul-NC formulation in pigs.

### 1.4 Discussion

One of the major unmet clinical needs in the management of inflammatory bowel diseases (IBD) is the management of Crohn's patients with anastomotic strictures. Within 20 years of diagnosis, up to 54% of Crohn's patients will develop fibrosis strictures. Le Berre et al., 2020. Endoscopic dilation is possible only in select cases, where the rest require surgery with ensuing loss of bowel, morbidity and mortality. Strictures, however, almost invariably recur, resulting in further surgery, abdominal adhesions, loss of bowel, short gut and/or other complications. Latella and Rieder, 2017. The seemingly immovable clinical evolution of these patients results, at least in part, from the lack of effective antifibrotic medications.

Recently, a number of antifibrotic candidate drugs have been identified through high throughput screens on hundreds to thousands of compounds. The drugs include itraconazole (chemically modified to CBR-096-4), Bollong et al., 2017, haloperidol, Rehman et al., 2019, and several other azole antifungal agents (oxiconazole, clotrimazole, and butoconazole). Braun et al., 2020. To our knowledge, however, there has not been validation of antifibrotic drugs in the GI tract, nor has there been a focus on formulation for local treatment. While it is currently unclear the mechanism by which sulconazole exerts antifibrotic effects, the known fungicidal action appears to be mediated through inhibition of fungal cytochrome P450 enzymes. Fromtling, 1988; Georgopapadakou and Walsh, 1996; Monk et al., 2020; Vanden Bossche et al., 1988.

In human lymphoblastoid and liver cells, sulconazole and other antifungal imidazole derivatives showed non-specific inhibition of P450 enzymes. Zhang et al., 2002. There is evidence suggesting that inhibition or down-regulation of P450 may have protective effect against tissue damage and inflammation induced by toxic chemicals such as ethanol and carbon tetrachloride. Nagappan et al., 2019; Song et al, 2021. P450 enzymes, however, play a critical role in drug metabolism and detoxification, so global inhibition is undesirable and would be associated with systemic side effects, further motivating formulation for localized treatment.

Some early efforts toward localized treatment to prevent fibrosis in the GI tract involved injection of anti-inflammatory agents, such as triamcinolone, East et al., 2007, or antifibrotic nucleic acids, Suzuki and Yoneyama, 2017, directly into the inflamed tissue. These attempts, however, did not result in clinically meaningful results, likely because a single injection of a drug or nucleic acids in solution would have a very short duration of action before being cleared from the body. In contrast, tissue remodeling processes like fibrosis occur over days and weeks, necessitating the development of a sustained-release formulation.

Because sulconazole has low water solubility, however, it is amenable to various approaches for sustained drug release, including encapsulation into polymer matrices. When considering the limitations on the volume that can be injected directly into intestinal tissue and the need for loading the highest amount of drug possible to achieve the longest duration of therapeutic effect, formulation as particulates with pure drug cores can achieve higher drug loadings than encapsulation. Hsueh et al., 2021; Farah et al., 2019. For example, the 500 mg/mL Sul-NC formulation described herein would contain approximately 96% drug loading by weight. Reduced amounts of excipients also might be advantageous for minimizing the potential for injection site reactions or build-up of materials that also can drive a fibrotic response. Further, it was observed that increasing the drug concentration and reducing the injection volume led to an increase in the maximum tolerated dose for Sul-NC (approximately 1,250 mg/kg compared to 50 mg/kg for free sulconazole) with subcutaneous injection in mice, likely due to the decreased surface area for absorption and decreased dissolution rate. Hsueh et al., 2021.

While more in-depth pharmacokinetic and safety studies are required, there is a rationale for repurposing a drug typically employed with topical administration as an injectable for the GI tract. Prior pharmacokinetic studies showed that systemic absorption of topically applied sulconazole was much higher than other azoles, in the range of 8.7-11.3% of a 9 g dose (two 4.5 g doses 12 h apart). Franz and Lehman, 1988. Further, elimination via the feces was one of the primary clearance routes, suggesting that topical sulconazole dosing is already associated with relatively high levels of drug exposure in the intestines. Franz and Lehman, 1988. Further, the potential toxicity of sulconazole is likely associated with its accumulation in the liver, which is going to be much higher with systemic dosing. Liver and kidney function upon administration of Sul-NC were assessed. It was shown that doses up to 1,250 mg/kg did not cause detectable changes in blood levels of ALT, AST, BUN, and creatinine 7 days after injection.

The presently disclosed subject matter represents the first demonstration of successful nanocrystal formulation of sulconazole for local, sustained release, antifibrotic effect in the GI tract. One potential limitation of the study is that animal models of GI tract fibrosis are few.

In addition, fibrosis in the GI tract is initiated by a variety of stimuli. To alleviate some of these concerns, however, the efficacy of Sul-NC was tested here in three animal models where fibrosis was induced by different inciting factors, i.e., chemical (the skin bleomycin model), ischemia (bowel transplantation model) and thermal (swine GI stricture model). While none of these models are a perfect representation of strictures in the GI tract in patients, the fact that Sul-NC was effective across these models that included different organ systems and inciting factors, is reassuring and creates the foundation for further studies that are aimed at specific etiologies for fibrosis in the GI tract. In addition, although maximum tolerated dose experiments were performed in rodents, further work should be focused on similar experiments following injection into the GI tract, since the systemic absorption may be different vs injection in the subcutaneous space. Therefore, a scaled up systemic preclinical study of its pharmacokinetics, toxicity, maximum tolerated dose is warranted as future work.

### 1.5 Summary

There is a dearth of drugs available for the prevention of pathological fibrosis. There are several indications, including intestinal structuring in IBD, in which local injection into the tissue can provide therapy with reduced risk of systemic side effects. To make an impact on disease processes that evolve over weeks and months, however, sustained-release approaches are needed. The presently disclosed subject matter demonstrates that sulconazole, an antifungal drug, displays potent antifibrotic character. A sulconazole nanocrystal formulation (Sul-NC) was engineered and a dramatic increase in the maximum tolerated dose, as well as efficacy in prevention of fibrosis in mouse models of skin and intestine tissue fibrosis and a patient-like pig esophagus stricture model was confirmed. It is expected that a full understanding of the pharmacokinetics, toxicity, MTD, and mechanism of action of the Sul-NC formulation would repurpose it as an antifibrotic drug for clinical trials.

### 1.6 Materials and Methods

1.6.1 Materials Human primary colonic fibroblast cells (CCD-18Co, CRL-1459) were purchase from the ATCC (Manassas, VA). Human liver stellate cells (LX2) were a gift from Dr. SL Friedman. Xu et al., 2005. TGF-β, rapid equilibrium dialysis (RED) device inserts (8K MWCO), PBS, TBS, ultra-pure water, ReverAid First Strand cDNA synthesis kit (1622), Pierce BCA protein Assay Kit (23227), DAPI (D1306), DMEM, alpha-MEM, FBS, 100 μm sterile cell strainers (22363549), tris base powder (BP152-500), pen-strep, and SYBR Green PCR Master Mix kit were obtained from Thermo Fisher Scientific (Waltham, MA, USA). α-SMA antibody (C-6198), polyethylene glycol 300 (PEG300) (81160), trifluoroacetic acid, and Tween 80 were purchased from Sigma Aldrich (St. Louis, MO, USA). Type 1 collagen antibody (ab138492) and anti-α-SMA antibody (ab5694) were obtained from Abcam (Waltham, MA). Tylose MH 300 (93800) was procured from Millipore Sigma (St. Louis, MO). High-performance liquid chromatography (HPLC) grade acetonitrile and water were purchased from Fisher Scientific (Hampton, NH, USA). TRIzol Reagent (15596026), Normal goat serum (31873) was obtained from Invitrogen (Waltham, MA, USA). RIPA buffer (9806) was obtained from Cell Signaling Technology (Danvers, MA, USA). Complete protease inhibitor cocktail (1183617001) was obtained from Roche. Mini-Protean TGX gel (4-15%), 1× Tris/Glycine/SDS Buffer (161-0772), nitrocellulose membrane, 1× Tris/Glycine with methanol (161-0771) were obtained from Bio-Rad Laboratories (Hercules, CA, USA). Intercept (TBS) blocking buffer (927-60001), IRDye® 800CW Goat anti-Rabbit IgG Secondary antibody (RRID AB_2651127) and IRDye® 680RD Goat anti-Mouse IgG Secondary antibody were purchased from Li-Cor (Lincoln, NE, USA). Sulconazole nitrate (K466) was purchased from AK Scientific (Union City, CA, USA). Pluronic F127 (Kolliphor P407) was purchased from BASF (Geismar, LA). Poly(vinyl alcohol) (PVA, 78 kDa, 88 mole % hydrolyzed) and uranyl acetate (98%, ACS reagent) was purchased from Polysciences Inc. (Warrington, PA). hydroxypropyl methylcellulose (HPMC, 3,550 mPa·s, USP grade HY124), and carboxymethyl cellulose sodium salt (CMC, 173 kDa) was purchased from Spectrum (Gardena, CA). Hyaluronic acid sodium salt (HA, 1-2 MDa) was purchased from Carbosynth (San Diego, CA). Ultra-thin (UL) carbon coated 400 mesh copper grids (EMS CF400-Cu-UL) were purchased from Electron Microscopy Sciences (Hatfield, PA). 0.5-mm zirconium oxide beads were purchased from Next Advance (Troy, NY). 2.0-mL Eppendorf tubes (T20-100) were obtained from stellar scientific. PCR primers were bought from IDT (Coralville, Iowa, USA). Insulin syringe was bought from BD (Franklin Lakes, NJ). Bleomycin (B3972) and pirfenidone (TCP1871) were bought from TCI (Portland, OR, USA), drug library was obtained from Johns Hopkins University.

### 1.6.2 Cell Culture

CCD-18Co cells were cultured according to the manufacturer's protocol using alpha-MEM supplemented with 10% FBS and 1% penicillin-streptomycin. The LX2 cell line was maintained in DMEM high glucose supplemented with 10% FBS and 1% penicillin-streptomycin. Cells were tested for mycoplasma, Young et al., 2010, before each experiment. Cell lines were maintained in 37° C. humid incubator supplied with 5% CO₂.

### 1.6.3 High Throughput Microscopy-Based Drug Screening

Human primary colonic fibroblasts (CCD-18Co) were seeded in 96 well flat bottom plate. Twenty-four hours later, cells were washed and starved with alpha-MEM medium without FBS for 24 h. The cells were then washed and the media was replaced with alpha-MEM supplemented with 10% FBS containing 5 ng/ml TGF-β and activated for 48 h. The cells were then washed again and fresh medium containing 5 ng/ml TGF-β and 10 μM of a drug from a library of 1,586 FDA approved small molecules was applied to the cells. After 4 days, the cells were fixed with 10% neutral formalin for 15 min and permeabilized with 0.5% Triton X-100 in PBS. The cells were then blocked for 1 h in 10% goat serum and stained for α-SMA, and cell nuclei. A Keyence BZ-X700 high throughput microscope was used for the high-throughput immunofluorescence scanning. All drugs in the library were initially screened in CCO-18Co cells at a concentration of 10 μM to evaluate effect on α-SMA production. Drugs that caused a visible reduction in fluorescent signal were then screened again at 5 μM and 10 μM to evaluate effect on both α-SMA and type 1 collagen production. The software (Keyence Bz-X700 Analyzer software) was programmed to capture 3 sets of images from each well, including the red channel of Alexa Fluor 594 (α-SMA), green channel of Alexa Fluor 488 (type 1 collagen), and blue channel-DAPI (nuclear) at a fixed exposure and exposure time. Images were analyzed with Image J2 Fiji (NIH, Bethesda, MD) to determine the drug treatment efficacy as assessed by reduction in the intensity of α-SMA and collagen staining per cell. The three images were averaged for each drug condition.

### 1.6.4 Western Blot

LX2 and CCD-18Co were seeded in 6 well plate. Twenty-four hours later, cells were washed and starved with Alpha MEM medium without FBS for 24 h. The cells were then washed and the media was replaced with Alpha MEM supplemented with 10% FBS containing 5 ng/ml TGF-β and activated for 48 h. The cells were then washed again and then incubated fresh medium containing 5 ng/ml TGF-0 and 10 μM sulconazole (dissolved in DMSO at 100 mM then diluted in cell culture media to 10 μM) for 96 h. The cells were then washed three times with cold PBS and treated with protein lysis RIPA buffer with complete protease inhibitor cocktail. Protein concentration was determined with Pierce BCA protein Assay Kit. 20 μg of total protein lysate from each treatment group was loaded into each well of a Mini-Protean TGX gel (4-15%), and electrophoresis was performed using 1× Tris/Glycine/SDS Buffer. Proteins were then transferred to nitrocellulose membranes using 1× Tris/Glycine with methanol. Membranes were blocked with Intercept (TBS) blocking buffer for 1 h at room temperature and then incubated with anti-α-SMA antibody (1:1000), and COL1A1 antibody (1:1000) at 4° C. overnight. Membranes were washed with TBS-0.1% Tween three times for 10 min each, and then incubated in IRDye-Secondary antibodies (1:10,000) at room temperature for 1 h. The membranes were then washed with TBS-0.1% Tween three times for 10 min each and rinsed with 1×TBS to remove residual Tween20. Membranes were then scanned with an Odyssey Image System (Li-Cor, Lincoln, NE, USA).

### 1.6.5 Development and Characterization of Sulconazole Nanocrystals (Sul-NC)

The Sul-NC was formulated using a wet bead-nanomilling method previously described. Hoang et al., 2019; Date et al., 2018; Date et al., 2021. The wet-bead milling was carried out using a lab-scale tissue homogenizer (TissueLyser LT, Qiagen Inc, Germantown, MD). Various stabilizers were employed to determine optimal sulconazole formulation, i.e. a formulation that resulted in particles that were relatively uniform in size, stable at room temperature, and injectable through a small gauge endoscopy needle. These stabilizers included polyvinyl alcohol (PVA), hyaluronic acid (HA), carboxymethylcellulose (CMC), hydroxypropyl methylcellulose (HPMC), and Pluronic® F127 (F127) (Table 1). The final formulation approach for animal dosing incorporated 500-mg sulconazole, 2.0 g of 0.5-mm zirconium oxide beads, and 1 mL of 2% (w/v) Pluronic F127 solution in a 2-mL Eppendorf tube. The contents were milled for 10 hours at a speed of 3000 oscillations per minute in a 4° C. cold room. The mixture was then passed through a 100-μm cell strainer to isolate the milling beads. Milling time was optimized to obtain particles around 200 nm, which was 5 hours for the final formulation. For pig studies, 30 tubes were prepared and then combined prior to characterization. Particle size, polydispersity index (PDI), and surface charge (ζ-potential) of the Sul-NC were measured using a Malvern Zetasizer Nano ZS (1730 scattering angle) (Malvern, Westborough, MA). For the particle size and polydispersity index measurement, Sul-NC were diluted 1:100 in ultrapure water and for the ζ-potential measurement, Sul-NC were diluted 1:40 in 10 mM NaCl (pH 7). The morphology of the Sul-NC was determined by the transmission electron microscopy (TEM). Sul-NC (500 mg/mL, 8 μL) was adsorbed to glow discharged (EMS GloQube, Hatfield, PA) ultra-thin (UL) carbon coated 400 mesh copper grids (EMS CF400-Cu-UL), by floatation for 2 min. Grids were rinsed in 3 drops (approximately 40 μL per drop, 1 min each) of tris-buffered saline (TBS) and negatively stained in 2 consecutive drops of 1% uranyl acetate with tylose (UAT), and quickly aspirated. Grids were imaged on a Hitachi 7600 TEM (Tokyo, Japan) (or Philips CM120 (Cambridge, MA)) operating at 80 kV with an AMT XR80 CCD detector (8 megapixel) (Wobum, MA). For stability studies, 500-mg/mL sulconazole samples were left on the benchtop (room temperature) or in the refrigerator (4° C.) for up to 112 days. Size was measured as described above periodically to assess particle stability.

### 1.6.5 In Vitro Drug Release

Sul-NC (5 mg/mL, 50 μL) were placed in a rapid equilibrium dialysis s(RED) device with an 8 kDa molecular weight cutoff (n=3). The outer reservoir was filled with 1 mL of 0.5% Tween 80 in phosphate buffered saline (PBS-T) solution. The samples were incubated on an orbital shaker with temperature control at 37° C. and 300 rpm. Every 24 h, 1 mL of the solution was collected and replenished with fresh 1 mL PBS-T solution. For the free sulconazole, 50 μL of solution containing sulconazole at the measured solubility limit (240 μg/mL) was added to the dialysis tubing and equilibrated with 1 mL of PBS-T solution in the outer reservoir (n=3). For quantifying the released sulconazole, the solution was transferred to an autosampler vial for high-performance liquid chromatography analysis (Prominence LC2030, Shimadzu, Columbia, MD). Separation was achieved with a Luna® 5 μm C18(2) 100 Å LC column 250×4.6 mm (Phenomenex, Torrance, CA) at room temperature using isocratic flow. Mobile phase A was water containing 0.1% trifluoroacetic acid (TFA) and mobile phase B was acetonitrile containing 0.1% TFA. The isocratic flow was composed 70% of mobile phase A and 30% mobile phase B at 1 mL/min flow rate for 10 min. The sulconazole retention time was 1.8 min with λmax=210 nm. A calibration curve for sulconazole was computed using the area under curve at RT=1.8 min over the range of 0.5-50 μg/mL. The drug amount was quantified and used to calculate the accumulation percentage. The release curves were plotted with GraphPad Prism 9 (San Diego, CA).

### 1.6.6 Animal Welfare Statement

All animal studies were approved by and conducted in accordance with the guidelines of the Institutional Animal Care and Use Committee at the Johns Hopkins University. All procedures were performed in compliance with the Guide for the Care and Use of Laboratory Animals and the Animal Welfare Act at an AAALAC-accredited facility. C57BL/6J (5-8 weeks old) male and female mice were obtained from Jackson Laboratory (Bar Harbor, ME). Yorkshire pigs (Sus scrofa domestica, female, 35-50 kg) were purchased from Archer Farms (Darlington, MD).

### 1.6.7 Maximum Tolerated Dose (MTD) Studies

5-8 week old male and female C57BL/6J mice were injected subcutaneously with increasing doses of free drug or Sul-NC until visible toxicity and/or mortality was observed. Each treatment group contain 3-9 mice. Free sulconazole was dissolved in DMSO at 100 mg/mL, then diluted into polyethylene glycol 300 (PEG300) to 10 mg/mL. Free sulconazole was injected at doses of 15 mg/kg, 30 mg/kg, 40 mg and 50 mg/kg via IP or SC injection (Table 3). Sul-NC was given subcutaneously (SC) at different concentrations and injection volumes with doses ranging from 250-2,500 mg/kg (Table 4). Mice were observed daily for up to 7 days after injection and were sacrificed if they became too visibly ill from drug toxicity. MTD was injected and blood were taken for liver and kidney function test (Table 5).

### 1.6.8 Skin Fibrosis Model

5-8 week old male C57BL/6J mice were utilized for bleomycin induced skin fibrosis model. The upper back was shaved, and 100 μL bleomycin (0.5 mg/ml in PBS) was injected subcutaneously every other day into one of five sectors in a defined 1-cm² area (see FIG. 3). Bleomycin injections were administered starting from position 1 to 5 cyclically for a period of 4 weeks. Chakraborty et al., 2020. The intestine transplanted mice were then randomly separated into 4 groups for the treatment; each group contain 3-5 mice. Treatments were administered as follows: free sulconazole 10 mg/kg in 50-μL PEG300 via IP injection every 2 days (14 total IP injections), 50-μL Sul-NC (50 mg/kg) SC once every 7 days (4 total SC injections), 50-μL Sul-NC (150 mg/kg) SC once every 7 days (4 total SC injections), or once daily oral pirfenidone (100 mg/kg in 100-μL PEG300). The free sulconazole dose was chosen to minimize chance of significant systemic toxicity while also repeatedly dosing to offset the rapid drug clearance. The Sul-NC were injected in sector 5 only. After 28 days, the dermis was collected for sectioning and staining by the Johns Hopkins Reference Histology Laboratory. Five representative images for each mouse were taken to quantify skin dermal thickness. For each image, five different areas were measured by a masked observer with image J Fiji (NIH, Bethesda, MD). Data were analyzed with GraphPad Prism 9 (San Diego, CA, USA).

### 1.6.9 Mouse Intestine Transplant Model

5-8 week old male C57BL/6J mice were utilized in an intestine transplantation model with minor modifications (FIG. 6). Meier et al., 2016. Briefly, a 6-cm segment of small intestine proximal to the ileocecal valve was excised from a donor mouse in a sterilized biosafety cabinet. The tissue was washed with cold saline 3 times, and carefully cut into 1 cm segments that were placed in a petri dish with cold saline. The back neck of the recipient mice was shaved. A small cut was made in the skin over the neck, and the donor intestine segment was placed in the subcutaneous pocket. The skin was then stitched with 3-0 nylon suture. Mice were given an intraperitoneal dose of cefazolin (300 mg/kg) for infection prevention. The intestine transplanted mice were then randomly separated into 4 groups for the treatment; each group contain 3-5 mice. The Sul-NC was injected subcutaneously (50 μL volume) adjacent to the transplanted tissue at 50 or 150 mg/kg using an insulin needle. As a positive control, pirfenidone was used at the dose previously shown to be effective in significantly reducing collagen deposition around the donor tissue (100 mg/kg in 100 μL PEG300 three times orally per day). Meier et al., 2016. After 7 days, the transplanted tissue was collected for sectioning and Masson's Trichrome staining by the Johns Hopkins Reference Histology Laboratory. Four representative images were taken for each tissue to quantify the collagen thickness. For each image, 10 different areas were measured by a masked observer with image J Fiji (NIH, Bethesda, MD). Data were analyzed with GraphPad Prism 9 (San Diego, CA, USA).

1.6.10 Quantitative RT-PCR (qRT-PCR)

RNA was extracted using TRIzol Reagent as previously described. Li et al., 2017. RNAs were reverse transcribed into cDNA with ReverAid First Strand cDNA synthesis kit. SYBR Green PCR Master Mix kit was utilized for the real time PCR using a QuantStudio 3 PCR machine (Applied Biosystems, Waltham, MA). Primers used are detailed in Table 6. Each reaction was performed in triplicate for each primer with a no-template negative control. QuantStudio Design and Analysis Software 1.5.2 was used to analyze the data using the ΔΔCT method.

TABLE 6
Primers used for RT-PCR

Homo sapiens actin alpha 2, smooth muscle (ACTA2)
Forward primer: CCGGGACTAAGACGGGAATC
Reverse primer: TTGTCACACACCAAGGCAGT
Homo sapiens glyceraldehyde-3-phosphate dehydrogenase (GAPDH)
Forward primer: GAAAGCCTGCCGGTGACTAA
Reverse primer: GCCCAATACGACCAAATCAGAG

### 1.6.11 Pig Esophagus Stricture Model

Argon plasma coagulation (APC) was performed as previously described. Li et al. 2021. Pigs were housed in the large animal facility at Johns Hopkins University School of Medicine and fed with a standard commercial swine diet and water was provided ad libitum. Pigs were acclimated for at least one week ahead of procedure.

Esophagogastroduodenoscopy (EGD) was performed using a forward-viewing single channel upper gastrointestinal endoscope with video scope system (EG-27i10 Video Gastroscope with 2.8 working channel Standard HD+, PENTAX Medical, USA). APC was performed with the APC electrosurgical device with APC generator (VIO 300D) and APC 2 unit (ERBE, Tübingen, Germany). Briefly, a 2 cm long portion of the circumferential mucosa was ablated to generate strictures at 30 cm, 40 cm, and 50 cm from the incisors. Li et al., 2021. In total, strictures were induced in 11 pigs, though 1 pig died of esophagus stricture perforation at 50 cm at day 12. Pigs were monitored daily by veterinary staff, and their weights were measured once per week. Once the pigs could not tolerate solid food, liquid diet (Ensure®) was supplied. Buprenorphine and carprofen were applied to control pain and fever as indicated by veterinary staff.

1.6.12 Esophagus Stricture Treatment with Local Sul-NC Injection Via EGD

Fourteen days after the APC procedure, pigs were sedated, and EGD with fluoroscopy was applied for internal evaluation of the stricture formation and measurement of the lumen diameters. Of the 30 ablated regions in the 10 pigs, 23 strictures were smaller than 6 mm and were included in the further analyses. Pigs were randomly assigned to either the vehicle control (n=5 pigs) or treatment (n=5 pigs) groups, such that the 23 strictures included in the study were split into 12 in the control group and 9 in the Sul-NC group. Strictures were then balloon dilated to 10 mm as previously described, Li et al., 2021, and either injected with Vehicle (2% F127) or Sul-NC. In the first two pigs (n=3 strictures) that received Sul-NC, 5 mL at 500 mg/mL concentration was split into 4×1.25 mL injections circumferentially around each stricture site (total dose 150 mg/kg). It was found that this concentration was challenging to push through the endoscopy needle, and the nanocrystals clogged the needle multiple times. Additionally, there was immediate fluid leakage from the tissue post injection. Thus, different dilutions were evaluated in vitro to determine a maximum concentration that could be used and easily administered through an endoscopy needle. It was found that diluting the Sul-NC formulation with additional 2% F127 to 300 mg/mL eliminated the needle clogging effect. Thus, the remaining 3 pigs (n=6 strictures) were injected with 300-mg/mL Sul-NC with a total volume of 3 mL split into 6×0.5 mL injections circumferentially around each stricture site (total dose 54 mg/kg). For the control pigs, 2% F127 was split into 6×0.5 mL injections circumferentially around each stricture site. Fourteen days after the treatment, pigs were sedated, the endoscope was positioned in the esophagus just proximal to the stricture, Omnipaque 240 was then injected through a catheter advanced through the scope and fluoroscopy images were obtained for evaluation of the stricture re-formation. Subsequently, EGD with fluoroscopy was applied to internally evaluate the stricture re-formation and measure the lumen diameters. Some strictures were so narrow that the EGD could not pass through, therefore pigs were euthanized and the esophagi were taken out to measure the stricture diameters ex vivo. Additionally, healthy tissue specimens were collected from uninvolved regions of the esophagus. Tissue specimens were placed in 10% neutral buffered formalin prior to paraffin embedding, sectioning (4 mm), and staining by the Johns Hopkins Reference Histology Laboratory. Slides were stained with either hematoxylin and eosin (H&E), Masson's Trichrome, or picrosirius red following standard protocols.

### 1.6.14 Statistical Analysis

Data are shown as mean±SD or mean±SEM for each graphical representation. Graphs were generated with GraphPad Prism 9. A two-tailed Student's t test (for comparison of two groups) or one-way analysis of variance (ANOVA) test (for comparison of more than two groups) were utilized to determine statistical significance with P value. Statistically significance was assumed if P<0.05 or 0.01.

### 1.6.14 Abbreviations

gastrointestinal tract—GI, inflammatory bowel disease—IBD, Crohn's disease—CD, ulcerative colitis—UC, extracellular matrix—ECM, transforming growth factor—TGF-β; alpha-smooth muscle actin—α-SMA, Sulconazole-nanocrystal—sul-NC; EGD—Esophagogastroduodenoscopy—EGD, Argon plasma coagulation—APC, generally regarded as safe—GRAS, polyvinyl alcohol—PVA, hyaluronic acid—HA, carboxymethylcellulose—CMC, Pluronic F127—F127, Transmission electron microscopy—TEM, maximum tolerated dose—MTD, intraperitoneal—IP, alanine aminotransferase—ALT, aspartate aminotransferase—AST, blood urea nitrogen—BUN, polydispersity index—PDI, trifluoroacetic acid—TFA, rapid equilibrium dialysis—RED

### Example 2

### Formulation of Sulconazole Nanocrystals

### 2.1 Methods

Sulconazole nanocrystals (Sul-NC) were formulated using a wet bead-based milling method. The nanomilling was carried out using a lab-scale tissue homogenizer (TissueLyser LT, Qiagen Inc, Germantown, MD). Various stabilizers were employed for formulation, including polyvinyl alcohol (PVA, 78 kDa, 88 mole % hydrolyzed), hyaluronic acid (HA, 1-2 MDa), hydroxypropyl methylcellulose (HPMC 3,550 mPa·s), and Pluronic F127 (Kolliphor P407, F127). All formulations included sulconazole (50-500 mg/mL as indicated), 2.0 g of 0.5 mm zirconium oxide beads, and 1 mL of stabilizer solution in a 2 mL Eppendorf tube. The contents were milled for 10 h at a speed of 3,000 oscillations/min in a 4° C. cold room. The mixture was then passed through a 100-μm cell strainer to isolate the milling beads. Particle size, polydispersity index (PDI), and surface charge (ζ-potential) of the Sul-NC were measured using a Malvern Zetasizer Nano ZS (173° scattering angle) (Malvern, Westborough, MA). For the particle size and polydispersity index measurement, Sul-NC were diluted 1:100 in ultrapure water and for the ζ-potential measurement, Sul-NC were diluted 1:40 in 10 mM NaCl (pH 7). Particles formulated in HA, PVA, and HPMC were stored at 4° C. during stability testing. To lyophilize particles, samples were either processed undiluted or after 1:10 dilution with water or 2% F127 as indicated, flash frozen in liquid nitrogen, and then placed in a 750 mL glass lyophilization flask (Flask no. 7542700, Labconco, Kansas City, MO) attached to a Labconco Freezone 4.5 Plus lyophilizer (Kansas City, MO). Samples were kept on the lyophilizer for 24 h at −74° C. and 0.110 mBar. As indicated, lyophilized samples were reconstituted in water prior to characterization for size, PDI, and ζ-potential as indicated above.

### 2.2 Results

Sul-NC formulated at 500 mg/mL in 2% F127, stored at either room temperature or 4° C., was measured for size, zeta potential, and PDI over 168 days (FIG. 10). The particles showed good stability through day 112, after which ζ-potential and PDI began to fluctuate suggesting potential metastability (overall particle size remained relatively stable).

Alternatively, the Sul-NC was left undiluted or diluted in either water or 2% F127 (w/w) and lyophilized, then reconstituted in water. Samples diluted in water had a smaller standard deviation in size and PDI than those diluted in F127, suggesting that dilution in water might lead to increased stability. Samples that were not diluted before lyphilization, however, had a size with the smallest standard deviation overall, PDI around 0.3, and near neutral zeta potential. This result suggests that it might be optimal to not dilute particles prior to lyophilization for the best stability outcomes after reconstitution (FIG. 11). Alternatively, the use of cryoprotectants may even further increase the particle stability through the lyophilization process.

Other excipients also were amenable to formulating sulconazole particles, though of generally larger average diameters. Formulating sulconazole with HA yielded large particles with a more negative zeta potential due to the anionic nature of HA (FIG. 12). Higher HA concentrations led to larger particle sizes and more instability, potentially due to the use of HA with high molecular weight. Smaller particle sizes may be obtained with lower molecular weight HA as a stabilizer. Particles milled with 5% HPMC were stable through day 14 of storage, where lower sulconazole concentrations 50 mg/mL and 100 mg/mL appeared more stable than the 200 mg/mL formulation after day 14 (FIG. 13). Formulating sulconazole with 5% PVA also yielded stable particles around 400 nm in size with near neutral zeta potential through day 14, where similarly, the lower sulconazole concentrations of 50 mg/mL and 100 mg/mL showed some improved stability compared to 200-mg/mL sulconazole (FIG. 14).

### Example 3

### Preparation and Characterization of Sulconazole Microcrystals (Sul-MCs)

Sulconazole nitrate (25 mg/mL) was weighed out and dissolved in acetonitrile. The suspension was heated at 75° C. until fully dissolved then cooled back to room temperature before drying and recrystallization under vacuum for 24 hrs. Sulconazole crystals were milled using wet-bead milling at 25-40 OS/second for 5-30 mins in the presence of 1.0-mm Zr beads using a TissueLyser (TissueLyser LT, Qiagen Inc, Germantown, MD). Various stabilizers were screened to assess the effect on sulconazole microcrystal (Sul-MC) size uniformity, ease in redispersion, sedimentation, and injectability through a small-gauge needle. The stabilizers screened included different concentrations and different molecular weights of carboxymethylcellulose (CMC), hydroxypropyl methylcellulose (HPMC), hydroxyethyl cellulose (HEC), polyvinyl alcohol (PVA), hyaluronic acid (HA), and sodium cholate (CHA) as small-molecule surfactant (Table 7 and Table 8). The mixture was then passed through a 100-μm cell strainer to isolate the milling beads. Particle size was measured using a Multisizer 4e Coulter Counter (Beckman coulter, IN, USA) and particle size and crystal structure was confirmed using scanning electron microscopy (SEM, MCP Thermo Scientific Helios G4 UC).

TABLE 7
Sul-MCs were wet-milled in the presence of different
stabilizers at different concentrations as shown.

| Composition | Milling time | Milling speed | Size |
|---|---|---|---|
| (% w/v) | (min) | (OS/sec) | (μm) |
| 1.25% CMC | 5 | 25 | 4.6 ± 2.1 |
| 2.5% CMC | 5 | 25 | 2.7 ± 0.3 |
| 0.25% HPMC | 5 | 25 | 11.8 ± 3.1 |
| 0.5% HPMC | 5 | 25 | 9.9 ± 2.7 |
| 0.5% HEC | 5 | 25 | 15.3 ± 4.1 |
| 1% HEC | 5 | 25 | 4.3 ± 0.5 |
| 1% PVA | 5 | 25 | 16.9 ± 3.6 |
| 1% PVA + 1% CHA | 5 | 25 | 15.9 ± 1.6 |

CMC, carboxymethyl cellulose;
HPMC, hydroxypropyl methylcellulose;
HEC, hydroxyethyl cellulose;
PVA, polyvinyl alcohol;
CHA, sodium cholate
Data presented as mean ± SD, n = 3.

Various cellulose derivatives (CMC, HPMC, HEC) were evaluated as viscosity builders to stabilize Sul-MCs at different concentrations using wet-bead milling (Table 7). The resulting Sul-MCs varied in size. Without wishing to be bound to any one particular theory, it is thought that a higher concentration of stabilizer resulted in smaller Sul-MC particle size. This observation could be due to higher attrition force imparted by beads in more viscous dispersion medium. Despite being in the order of desired size range for injection through a small gauge needle, Sul-MCs milled in the presence of cellulose derivatives or PVA resulted in the formation of sediment that was not easily dispersible and was not easily passed through a small gauge needle within 24 hr.

TABLE 8
Wet-bead milling of Sul-MCs in the presence of different
molecular weight HA at different concentration ranges.

| Composition | Milling time | Milling speed | Size |
|---|---|---|---|
| (% w/v) | (min) | (OS/sec) | (μm) |
| 0.1% HA (2.5 mDa) | 10 | 25 | 18.5 ± 1.2 |
| 0.1% HA (2.5 mDa) + | 10 | 25 | 13.6 ± 5.5 |
| 1% CHA | | | |
| 0.05% HA (2.5 mDa) | 10 | 25 | 15.5 ± 0.9 |
| 0.05% HA (2.5 mDa) + | 10 | 25 | 27.1 ± 3.0 |
| 1% CHA | | | |
| 5% HA (5 kDa) | 10 | 25 | 9.5 ± 1.4 |
| 1% HA (100 kDa) | 10 | 25 | 21.8 ± 5.7 |
| 2% HA (100 kDa) | 10 | 25 | 15.4 ± 2.0 |
| 1% HA (500 kDa) | 10 | 25 | 18.0 ± 0.8 |
| 0.5% HA (500 kDa) | 10 | 25 | 25.9 ± 0.7 |
| 0.25% HA (500 kDa) | 20 | 25 | 17.7 ± 2.0 |
| 0.25% HA (500 kDa) + | 20 | 25 | 18.0 ± 1.6 |
| 0.1% CHA | | | |
| 0.5% HA (100 kDa) | 20 | 25 | 13.8 ± 1.7 |
| 0.5% HA (10 kDa) + | 20 | 25 | 21.8 ± 1.6 |
| 0.1% CHA | | | |
| 0.5% HA (100 kDa) | 20 | 50 | 6.6 ± 1.0 |
| 0.5% HA (100 kDa) + | 20 | 50 | 14.3 ± 2.8 |
| 0.1% CHA | | | |
| 0.25% HA (500 kDa) (R1) | 30 | 40 | 5.5 ± 0.03 |
| 0.25% HA (500 kDa) + | 30 | 40 | 11.9 ± 0.4 |
| 0.1% CHA (R1) | | | |
| 0.25% HA (500 kDa) (R2) | 30 | 40 | 5.8 ± 0.7 |
| 0.25% HA (500 kDa) + | 30 | 40 | 15.8 ± 0.8 |
| 0.1% CHA (R2) | | | |
| 0.5% HA (100 kDa) | 30 | 40 | 6.7 ± 0.8 |
| 0.5% HA (100 kDa) + | 30 | 40 | 17.9 ± 1.4 |
| 0.1% CHA | | | |

HA, hyaluronic acid;
CHA, sodium cholate;
R1, repeat 1;
R2, repeat 2
Data presented as mean ± SD, n = 3.

HA was screened at various molecular weights as a stabilizer for Sul-MCs during wet-bead milling with or without small molecule surfactant CHA (Table 8). While higher molecular weight HA (e.g., 2.5 mDa) resulted in Sul-MC in an acceptable size range, the resulting dispersion was very difficult to pass through small gauge (26 G) needle for injection. Further, phase separation was observed during storage at 4° C. after 24 hrs.

Accordingly, lower molecular weight HA (e.g., 100 kDa or 500 kDa) was tested at different concentration ranges. Increasing the HA concentration resulted in reduced Sul-MC particle size, which could be due to higher viscosity. Similarly, addition of CHA reduced the viscosity of the medium, resulting in increased Sul-MC particle size. When the milling time (30 min) and oscillation cycle (40OS) was increased, a stable Sul-MC formulation with a desired size range was observed. Moreover, the Sul-MCs easily passed through small gauge needle for administration. Furthermore, size and crystal structure of these Sul-MCs were also confirmed via SEM images. Importantly, the Sul-MC particle size was reproducible across two replicates (R1 and R2) as shown in Table 8.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Rieder F, Brenmoehl J, Leeb S, et al. Wound healing and fibrosis in intestinal disease. Gut 2007; 56:130-9.
Henderson N C, Rieder F, Wynn T A. Fibrosis: from mechanisms to medicines. Nature 2020; 587:555-566.
Rieder F. The gut microbiome in intestinal fibrosis: environmental protector or provocateur? Sci Transl Med 2013; 5:190ps10.
Rieder F, Fiocchi C. Intestinal fibrosis in IBD—a dynamic, multifactorial process. Nat Rev Gastroenterol Hepatol 2009; 6:228-35.
Bollong M J, Yang B, Vergani N, et al. Small molecule-mediated inhibition of myofibroblast transdifferentiation for the treatment of fibrosis. Proc Natl Acad Sci USA 2017; 114:4679-4684.
Rosenbloom J, Mendoza F A, Jimenez S A. Strategies for anti-fibrotic therapies. Biochim Biophys Acta 2013; 1832:1088-103.
Raghu G, Selman M. Nintedanib and pirfenidone. New antifibrotic treatments indicated for idiopathic pulmonary fibrosis offer hopes and raises questions. Am J Respir Crit Care Med 2015; 191:252-4.
Latella G, Rieder F. Intestinal fibrosis: ready to be reversed. Current Opinion in Gastroenterology 2017; 33:239-245.
Dahlhamer J M, Zammitti E P, Ward B W, et al. Prevalence of Inflammatory Bowel Disease Among Adults Aged >/=18 Years—United States, 2015. MMWR Morb Mortal Wkly Rep 2016; 65:1166-1169.
Schmoyer C J, Saidman J, Bohl J L, et al. The Pathogenesis and Clinical Management of Stricturing Crohn Disease. Inflamm Bowel Dis 2021.
Le Berre C, Ananthakrishnan A N, Danese S, et al. Ulcerative Colitis and Crohn's Disease Have Similar Burden and Goals for Treatment. Clin Gastroenterol Hepatol 2020; 18:14-23.
Lan N, Stocchi L, Ashburn J H, et al. Outcomes of Endoscopic Balloon Dilation vs Surgical Resection for Primary Ileocolic Strictures in Patients With Crohn's Disease. Clin Gastroenterol Hepatol 2018; 16:1260-1267.
Cosnes J, Nion-Larmurier I, Beaugerie L, et al. Impact of the increasing use of immunosuppressants in Crohn's disease on the need for intestinal surgery. Gut 2005; 54:237-41.
D'Amico F, Pugliese N, Peyrin-Biroulet L, et al. Efficacy of anti-TNFalpha drugs in patients with stricturing Crohn's disease. Expert Rev Gastroenterol Hepatol 2020; 14:347-353.
Li J, Mao R, Kurada S, et al. Pathogenesis of fibrostenosing Crohn's disease. Transl Res 2019; 209:39-54.
Moschen A R, Tilg H, Raine T. IL-12, IL-23 and IL-17 in IBD: immunobiology and therapeutic targeting. Nat Rev Gastroenterol Hepatol 2019; 16:185-196.
Madadi-Sanjani O, Zimmer J, Gosemann J H, et al. Topical Mitomycin C Application in Pediatric Patients with Recurrent Esophageal Strictures-Report on Unfavorable Results. Eur J Pediatr Surg 2018; 28:539-546.
East J E, Brooker J C, Rutter M D, et al. A pilot study of intrastricture steroid versus placebo injection after balloon dilatation of Crohn's strictures. Clin Gastroenterol Hepatol 2007; 5:1065-9.
Li L, Itani M I, Salimian K J, et al. A patient-like swine model of gastrointestinal fibrotic strictures for advancing therapeutics. Sci Rep 2021; 11:13344.
Hsueh H T, Kim Y C, Pitha I, et al. Ion-Complex Microcrystal Formulation Provides Sustained Delivery of a Multimodal Kinase Inhibitor from the Subconjunctival Space for Protection of Retinal Ganglion Cells. Pharmaceutics 2021; 13.
Yamamoto T, Takagawa S, Katayama I, et al. Animal model of sclerotic skin. I: Local injections of bleomycin induce sclerotic skin mimicking scleroderma. J Invest Dermatol 1999; 112:456-62.
Meier R, Lutz C, Cosin-Roger J, et al. Decreased Fibrogenesis After Treatment with Pirfenidone in a Newly Developed Mouse Model of Intestinal Fibrosis. Inflamm Bowel Dis 2016; 22:569-82.
Latella G, Rieder F. Intestinal fibrosis: ready to be reversed. Curr Opin Gastroenterol 2017; 33:239-245.
Rehman M, Vodret S, Braga L, et al. High-throughput screening discovers antifibrotic properties of haloperidol by hindering myofibroblast activation. JCI Insight 2019; 4.
Braun C B, Girtman M, Jenson P, et al. Inhibition of fibrosis with multi-agent therapy in pulmonary fibrosis: Results of a drug library screening. bioRxiv 2020; 2020.2006.2029.178061.
Fromtling R A. Overview of medically important antifungal azole derivatives. Clin Microbiol Rev 1988; 1:187-217.
Georgopapadakou N H, Walsh T J. Antifungal agents: chemotherapeutic targets and immunologic strategies. Antimicrob Agents Chemother 1996; 40:279-91.
Monk B C, Sagatova A A, Hosseini P, et al. Fungal Lanosterol 14alpha-demethylase: A target for next-generation antifungal design. Biochim Biophys Acta Proteins Protcom 2020; 1868:140206.
Vanden Bossche H, Marichal P, Gorrens J, et al. Mode of action studies. Basis for the search of new antifungal drugs. Ann N Y Acad Sci 1988; 544:191-207.
Zhang W, Ramamoorthy Y, Kilicarslan T, et al. Inhibition of cytochromes P450 by antifungal imidazole derivatives. Drug Metab Dispos 2002; 30:314-8.
Nagappan A, Kim J H, Jung D Y, et al. Cryptotanshinone from the Salvia miltiorrhiza Bunge Attenuates Ethanol-Induced Liver Injury by Activation of AMPK/SIRT1 and Nrf2 Signaling Pathways. Int J Mol Sci 2019; 21.
Song X, Cui W, Gao Z, et al. Structural characterization and amelioration of sulfated polysaccharides from Ganoderma applanatum residue against CCl4-induced hepatotoxicity. Int Immunopharmacol 2021; 96:107554.
Suzuki K, Yoneyama H. New endoscopic approach of anti-fibrotic therapy for inflammatory bowel disease. Ann Transl Med 2017; 5:191.
Farah S, Doloff J C, Muller P, et al. Long-term implant fibrosis prevention in rodents and non-human primates using crystallized drug formulations. Nat Mater 2019; 18:892-904.
Franz T J, Lehman P. Percutaneous absorption of sulconazole nitrate in humans. J Pharm Sci 1988; 77:489-91.
Akhurst R J, Hata A. Targeting the TGFbeta signalling pathway in disease. Nat Rev Drug Discov 2012; 11:790-811.
Choi H S, Kim J H, Kim S L, et al. Disruption of the NF-kappaB/IL-8 Signaling Axis by Sulconazole Inhibits Human Breast Cancer Stem Cell Formation. Cells 2019; 8.
Atreya I, Atreya R, Neurath M F. NF-kappaB in inflammatory bowel disease. J Intern Med 2008; 263:591-6.
Xu L, Hui A Y, Albanis E, et al. Human hepatic stellate cell lines, LX-1 and LX-2: new tools for analysis of hepatic fibrosis. Gut 2005; 54:142-51.
Young L, Sung J, Stacey G, et al. Detection of Mycoplasma in cell cultures. Nat Protoc 2010; 5:929-34.
Hoang T, Zierden H, Date A, et al. Development of a mucoinert progesterone nanosuspension for safer and more effective prevention of preterm birth. J Control Release 2019; 295:74-86.
Date A A, Halpert G, Babu T, et al. Mucus-penetrating budesonide nanosuspension enema for local treatment of inflammatory bowel disease. Biomaterials 2018; 185:97-105.
Date A A, Kates M, Yoshida T, et al. Preclinical evaluation of a hypotonic docetaxel nanosuspension formulation for intravesical treatment of non-muscle-invasive bladder cancer. Drug Deliv Transl Res 2021; 11:2085-2095.
Chakraborty D, Zhu H, Jungel A, et al. Fibroblast growth factor receptor 3 activates a network of profibrotic signaling pathways to promote fibrosis in systemic sclerosis. Sci Transl Med 2020; 12.
Li L, Piontek K, Ishida M, et al. Extracellular vesicles carry microRNA-195 to intrahepatic cholangiocarcinoma and improve survival in a rat model. Hepatology 2017; 65:501-514.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A formulation comprising a plurality of sulconazole nanocrystals and one or more stabilizers.

2. The formulation of claim 1, wherein the one or more stabilizers is selected from polyvinyl alcohol (PVA), hyaluronic acid (HA), carboxymethylcellulose (CMC), hydroxypropyl methylcellulose (HPMC), hydroxyethyl cellulose (HEC), sodium cholate (CHA), a cellulose derivative, a polysaccharide, polyethylene glycol, a poloxamer, and combinations thereof.

3. The formulation of claim 3, wherein the poloxamer comprises poloxamer 407.

4. The formulation of claim 1, wherein the concentration of sulconazole is between about 10 and about 500 mg/mL.

5. The formulation of claim 4, wherein the formulation comprises:
(a) between about 1.5% to about 5% PVA;
(b) between about 0.5% and 1% HA;
(c) between about 1% to about 2% CMC;
(d) between about 1% HPMC to about 5% HPMC; and
(e) between about 2% to about 6% poloxamer 407.

6. The formulation of claim 1, wherein the formulation is lyophilized.

7. A precursor formulation comprising the formulation of claim 1 and a plurality of milling beads.

8. The precursor formulation of claim 7, wherein the plurality of milling beads comprise zirconium oxide beads.

9. The precursor formulation of claim 7, wherein the formulation comprises about 500-mg sulconazole, about 2.0 g of 0.5-mm zirconium oxide beads, and about 1 mL of 2% (w/v) poloxamer 407.

10. A method for treating or preventing fibrosis or intestinal re-stricturing in a gastrointestinal (GI) tract of a subject in need of treatment thereof, the method comprising administering to the subject a formulation of claim 1.

11. The method of claim 10, wherein the administering of the formulation is via injection.

12. The method of claim 11, wherein the injection comprises an intraperitoneal (IP) or a subcutaneous (SC) injection.

13. The method of claim 11, wherein the formulation is injected in a proximity of a stricture site.

14. The method of claim 11, wherein the formulation is injected in a proximity of a stricture site after a surgical or endoscopic procedure.

15. The method of claim 10, wherein the fibrosis is associated with an inflammatory bowel disease (IBD).

16. The method of claim 15, wherein the inflammatory bowel disease is selected from Crohn's disease and (CD), ulcerative colitis (UC), and combinations thereof.

17. The method of claim 10, wherein the administration of the formulation:
modulates an acute healing response and/or interrupts one or more pathological fibrotic tissue remodeling processes;
comprises a decrease in a collagen layer thickness in a small intestine of the subject; and/or
results in a folded, flexible epithelial structure more similar to healthy tissue.

18-19. (canceled)

20. The method of claim 10, wherein the administration of the formulation is a sustained-release administration.

21. The method of claim 10, wherein a concentration of sulconazole in the formulation has a range between about 100 mg/mL and about 500 mg/mL.

22. The method of claim 10, wherein a volume of the formulation injected has a range between about 10 μL to about 100 μL.

23. The method of claim 10, wherein the formulation is injected with a dose of sulconazole between about 100 mg/kg and about 1875 mg/kg.
